SEQUENCE LISTING (114 sequences)

SEQ ID NO: 1 - DNA, 25 bp, Artificial Sequence
  Description of Artificial Sequence Synthetic primer
gaaaggtattcaaggagaatcaagt

SEQ ID NO: 2 - DNA, 34 bp, Artificial Sequence
  Description of Artificial Sequence Synthetic probe
aacctcaagcaactgaaacaacagaagcatcaca

SEQ ID NO: 3 - DNA, 25 bp, Artificial Sequence
  Description of Artificial Sequence Synthetic primer
ttgtattcacggatacctgtaccag

SEQ ID NO: 4 - DNA, 29 bp, Artificial Sequence
  Description of Artificial Sequence Synthetic primer
tgaaatattattagctgattcaggttacg

SEQ ID NO: 5 - DNA, 33 bp, Artificial Sequence
  Description of Artificial Sequence Synthetic probe
cccagtacagatcctttcaatctatagcgcatt

SEQ ID NO: 6 - DNA, 25 bp, Artificial Sequence
  Description of Artificial Sequence Synthetic primer
ccaaactttgtttttcgtgtctttt

SEQ ID NO: 7 - DNA, 25 bp, Artificial Sequence
  Description of Artificial Sequence Synthetic primer
aagttaataacttgtggataactgg

SEQ ID NO: 8 - DNA, 35 bp, Artificial Sequence
  Description of Artificial Sequence Synthetic probe
aattctgtatgaggagataataatttggagggtgt

SEQ ID NO: 9 - DNA, 23 bp, Artificial Sequence
  Description of Artificial Sequence Synthetic primer
cgtggatttaatgtccaccattt

SEQ ID NO: 10 - DNA, 35 bp, Artificial Sequence
  Description of Artificial Sequence Synthetic probe
aacaccctccaaattattatctcctcatacagaat

SEQ ID NO: 11 - DNA, 25 bp, Artificial Sequence
  Description of Artificial Sequence Synthetic primer
gtgtgaacaagttaataacttgtgg

SEQ ID NO: 12 - DNA, 35 bp, Artificial Sequence
  Description of Artificial Sequence Synthetic probe
atgaggagataataatttggagggtgttaaatggt

SEQ ID NO: 13 - DNA, 25 bp, Artificial Sequence
  Description of Artificial Sequence Synthetic primer
attgaatgaacgtggatttaatgtc

SEQ ID NO: 14 - DNA, 35 bp, Artificial Sequence
  Description of Artificial Sequence Synthetic probe
tccaccatttaacaccctccaaattattatctcct

SEQ ID NO: 15 - DNA, 25 bp, Artificial Sequence
  Description of Artificial Sequence Synthetic primer
atattgaatgaacgtggatttaatg

SEQ ID NO: 16 - DNA, 30 bp, Artificial Sequence
  Description of Artificial Sequence Synthetic probe
agataataatttggagggtgttaaatggtg

SEQ ID NO: 17 - DNA, 25 bp, Artificial Sequence
  Description of Artificial Sequence Synthetic primer
ttgttgaagatcatccgcaattcaa

SEQ ID NO: 18 - DNA, 27 bp, Artificial Sequence
  Description of Artificial Sequence Synthetic probe
atttatttacaatgggaaaaggtggcg

SEQ ID NO: 19 - DNA, 21 bp, Artificial Sequence
  Description of Artificial Sequence Synthetic primer
gttgctactgtcgttttacca

SEQ ID NO: 20 - DNA, 25 bp, Artificial Sequence
  Description of Artificial Sequence Synthetic primer
aagcacaatctatagaaccttatga

SEQ ID NO: 21 - DNA, 31 bp, Artificial Sequence
  Description of Artificial Sequence Synthetic probe
actgtacatgaaccacttaactgttctttca

SEQ ID NO: 22 - DNA, 25 bp, Artificial Sequence
  Description of Artificial Sequence Synthetic primer
tgtaaattcattaaatgctgctact

SEQ ID NO: 23 - DNA, 25 bp, Artificial Sequence
  Description of Artificial Sequence Synthetic primer
tgactttattgttgaagatcatccg

SEQ ID NO: 24 - DNA, 24 bp, Artificial Sequence
  Description of Artificial Sequence Synthetic primer
tgctgcagacgattataaagcaca

SEQ ID NO: 25 - DNA, 23 bp, Artificial Sequence
  Description of Artificial Sequence Synthetic primer
acctaatctctctatagccaatt

SEQ ID NO: 26 - DNA, 28 bp, Artificial Sequence
  Description of Artificial Sequence Synthetic probe
agattgtgctttataatcgtctgcagca

SEQ ID NO: 27 - DNA, 25 bp, Artificial Sequence
  Description of Artificial Sequence Synthetic primer
catgaaccacttaactgttctttca

SEQ ID NO: 28 - DNA, 25 bp, Artificial Sequence
  Description of Artificial Sequence Synthetic primer
attcttgttctaaagttttatcgga

SEQ ID NO: 29 - DNA, 24 bp, Artificial Sequence
  Description of Artificial Sequence Synthetic primer
gaaggttatttggcagaagagagc

SEQ ID NO: 30 - DNA, 28 bp, Artificial Sequence
  Description of Artificial Sequence Synthetic probe
tgcggcctccacttttcgtcttgtctag

SEQ ID NO: 31 - DNA, 22 bp, Artificial Sequence
  Description of Artificial Sequence Synthetic primer
gaaacgaaagcgctgtgtatcg

SEQ ID NO: 32 - DNA, 27 bp, Artificial Sequence
  Description of Artificial Sequence Synthetic probe
gctggagctagacaagacgaaaagtgg

SEQ ID NO: 33 - DNA, 28 bp, Artificial Sequence
  Description of Artificial Sequence Synthetic probe
ctccacttttcgtcttgtctagctccag

SEQ ID NO: 34 - DNA, 27 bp, Artificial Sequence
  Description of Artificial Sequence Synthetic probe
tttcgtcttgtctagctccagcgcctg

SEQ ID NO: 35 - DNA, 29 bp, Artificial Sequence
  Description of Artificial Sequence Synthetic probe
agacaagacgaaaagtggaggccgcaatg

SEQ ID NO: 36 - DNA, 24 bp, Artificial Sequence
  Description of Artificial Sequence Synthetic primer
tggagctagacaagacgaaaagtg

SEQ ID NO: 37 - DNA, 30 bp, Artificial Sequence
  Description of Artificial Sequence Synthetic probe
tgtcgatacacagcgctttcgtttcgttca

SEQ ID NO: 38 - DNA, 20 bp, Artificial Sequence
  Description of Artificial Sequence Synthetic primer
aacatgactcgttccctcaa

SEQ ID NO: 39 - DNA, 30 bp, Artificial Sequence
  Description of Artificial Sequence Synthetic probe
tgaacgaaacgaaagcgctgtgtatcgaca

SEQ ID NO: 40 - DNA, 25 bp, Artificial Sequence
  Description of Artificial Sequence Synthetic primer
ttttgtcattgagagaacatgactc

SEQ ID NO: 41 - DNA, 27 bp, Artificial Sequence
  Description of Artificial Sequence Synthetic probe
ggccaactgtcgatacacagcgctttc

SEQ ID NO: 42 - DNA, 32 bp, Artificial Sequence
  Description of Artificial Sequence Synthetic probe
tcaaaactgaacgaaacgaaagcgctgtgtat

SEQ ID NO: 43 - DNA, 24 bp, Artificial Sequence
  Description of Artificial Sequence Synthetic primer
gtcattgagagaacatgactcgtt

SEQ ID NO: 44 - DNA, 32 bp, Artificial Sequence
  Description of Artificial Sequence Synthetic probe
acacagcgctttcgtttcgttcagttttgagg

SEQ ID NO: 45 - DNA, 23 bp, Artificial Sequence
  Description of Artificial Sequence Synthetic primer
ttgagagaacatgactcgttccc

SEQ ID NO: 46 - DNA, 25 bp, Artificial Sequence
  Description of Artificial Sequence Synthetic primer
gagtcatgttctctcaatgacaaaa

SEQ ID NO: 47 - DNA, 31 bp, Artificial Sequence
  Description of Artificial Sequence Synthetic probe
ttgaaaactagataaccggaaaagcggaggc

SEQ ID NO: 48 - DNA, 20 bp, Artificial Sequence
  Description of Artificial Sequence Synthetic primer
cgaagaacatttgcccatcg

SEQ ID NO: 49 - DNA, 19 bp, Artificial Sequence
  Description of Artificial Sequence Synthetic primer
gcgctttcgtttcgttcag

SEQ ID NO: 50 - DNA, 34 bp, Artificial Sequence
  Description of Artificial Sequence Synthetic probe
tgagggaacgagtcatgttctctcaatgacaaaa

SEQ ID NO: 51 - DNA, 25 bp, Artificial Sequence
  Description of Artificial Sequence Synthetic primer
tccgcttttccggttatctagtttt

SEQ ID NO: 52 - DNA, 21 bp, Artificial Sequence
  Description of Artificial Sequence Synthetic primer
ttttgagggaacgagtcatgt

SEQ ID NO: 53 - DNA, 29 bp, Artificial Sequence
  Description of Artificial Sequence Synthetic probe
cggttatctagttttcaaggaacgatttt

SEQ ID NO: 54 - DNA, 34 bp, Artificial Sequence
  Description of Artificial Sequence Synthetic probe
gcttttccggttatctagttttcaaggaacgatt

SEQ ID NO: 55 - DNA, 34 bp, Artificial Sequence
  Description of Artificial Sequence Synthetic probe
atcgttccttgaaaactagataaccggaaaagcg

SEQ ID NO: 56 - DNA, 20 bp, Artificial Sequence
  Description of Artificial Sequence Synthetic primer
gccaactgtcgatacacagc

SEQ ID NO: 57 - DNA, 33 bp, Artificial Sequence
  Description of Artificial Sequence Synthetic probe
atgactcgttccctcaaaactgaacgaaacgaa

SEQ ID NO: 58 - DNA, 34 bp, Artificial Sequence
  Description of Artificial Sequence Synthetic probe
ttcgtttcgttcagttttgagggaacgagtcatg

SEQ ID NO: 59 - DNA, 25 bp, Artificial Sequence
  Description of Artificial Sequence Synthetic primer
ggttatctagttttcaaggaacgat

SEQ ID NO: 60 - DNA, 21 bp, Artificial Sequence
  Description of Artificial Sequence Synthetic primer
ctgtcgatacacagcgctttc

SEQ ID NO: 61 - DNA, 27 bp, Artificial Sequence
  Description of Artificial Sequence Synthetic probe
acgagtcatgttctctcaatgacaaaa

SEQ ID NO: 62 - DNA, 34 bp, Artificial Sequence
  Description of Artificial Sequence Synthetic probe
gtcattgagagaacatgactcgttccctcaaaac

SEQ ID NO: 63 - DNA, 33 bp, Artificial Sequence
  Description of Artificial Sequence Synthetic probe
tcgttcagttttgagggaacgagtcatgttctc

SEQ ID NO: 64 - DNA, 20 bp, Artificial Sequence
  Description of Artificial Sequence Synthetic primer
tacacagcgctttcgtttcg

SEQ ID NO: 65 - DNA, 23 bp, Artificial Sequence
  Description of Artificial Sequence Synthetic primer
gggaacgagtcatgttctctcaa

SEQ ID NO: 66 - DNA, 24 bp, Artificial Sequence
  Description of Artificial Sequence Synthetic primer
cgctttcgtttcgttcagttttga

SEQ ID NO: 67 - DNA, 21 bp, Artificial Sequence
  Description of Artificial Sequence Synthetic primer
ggctctcgaagccaaatgttc

SEQ ID NO: 68 - DNA, 28 bp, Artificial Sequence
  Description of Artificial Sequence Synthetic probe
ggctctcttctgccaaataaccttcccc

SEQ ID NO: 69 - DNA, 24 bp, Artificial Sequence
  Description of Artificial Sequence Synthetic primer
tccacttttcgtcttgtctagctc

SEQ ID NO: 70 - DNA, 21 bp, Artificial Sequence
  Description of Artificial Sequence Synthetic primer
cgaagccaaatgttcttcacc

SEQ ID NO: 71 - DNA, 21 bp, Artificial Sequence
  Description of Artificial Sequence Synthetic primer
agtcatgttctctcaatgaca

SEQ ID NO: 72 - DNA, 22 bp, Artificial Sequence
  Description of Artificial Sequence Synthetic primer
agtcatgttctctcaatgacaa

SEQ ID NO: 73 - DNA, 1731 bp, Staphylococcus sp.
atggagatggatgctgttaaatatttaaataaattgaatctagataacattgagttaacaaaatatttgttttttactggtaaaggtggcgtaggcaaaacgacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgctttctgaaatgaaagaacagttaagtggttcatgtacagttgaagtagcagcatttaatgaatttacaaattttttatccgataaaactttagaacaagaatttgatttcattatatttgatacagctccaacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctactcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatcgatgaaattgaaaatagtaaagttcaatatttatttacaatgggaaaaggtggcgttggtaaaacgacagtagcaacgcaattagctacaacgttatctaataaaggatatcgtgttcttttagcaactactgaccctactaaagaaattaatgttgaaactacaagtaatttaaatactgcttatattgatgaagaacaagcattagaaaaatataaaaaagaagtactagccacagtgaatgatgatacaccacaagacgatattgattatattatggaagatttaaaatcaccttgtacagaagaaatagcatttttcaaagcctttagtgacattatggagaatcaagacgacgtggattacgtcattgtagatacagctccaacaggccataccttgctgttacttgattctagtgaaaatcatcatagagaattaaagaaaaaatcaactcaaactaccagtaatgttgaaacattattacctaagattcaaaataaaaatttaacacagatgataatcgtaacactagcagaaaaaacaccttatttagaatctaaacgtttagtagaagatttaaatagagctaatataggccataattggtgggttgttaatcaatcgttagttacactaaatcaacgtgatgacctttttagtaacaaaaaagaagatgaatcattttggataaacaagattaaaaatgaaagtcttaataattactttgtcataccttatcgaatatcagaatattaa

SEQ ID NO: 74 - DNA, 1731 bp, Staphylococcus sp.
gtggagatggatgctgttaaatacttaaataaattgaacctaaataacattgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgctttctgaaatgaaagaacagttaagtggttcatgtacagttgaagtagcagcatttaatgaatttacaaattttttatccgataaaactttagaacaagaatttgatttcattatatttgatacagctcccacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctactcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatcgatgaaattgaaaatagtaaagttcaatatttatttacaatgggaaaaggtggcgttggtaaaacgacagtagcaacgcaattagctacaacgttatctaataaaggatatcgtgttcttttagcaactactgaccctactaaagaaattaatgttgaaaccacaagtaatttaaatactgcttatattgatgaagaacaagcattagaaaagtataaaaaagaagtactagccacagtgaatgatgatacaccacaagacgatatcgattatattatggaagatttaaaatcaccttgtacagaagaaatagcatttttcaaagcctttagtgacattatggagaatcaagacgacatggattacgtcattgtagatacagctcctacaggccatactttgcttttacttgattctagtgaaaatcatcatagagaattaaagaaaaaatcaactcaaactaccagtaatgttgaaacattattacctaagattcaaaataaaaatttaacacagatgataatcgtaacactagcagaaaaaacaccttatttagaatctaaacgtttagtagaagatttaaatagagctaatataggtcataattggtgggtcgttaatcaatcgttagttacgctaaatcaacgtgatgacctttttagtaacaaaaaagaagatgaatcattttggataaacaaaattaaaaatgaaagttttgataattactttgtcataccttatcgaatatcagaatgttaa

SEQ ID NO: 75 - DNA, 1731 bp, Staphylococcus sp.
gtggagatggatgctgttaagtacttaaataaattgaatttagataacgttgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgcttgctgagatgaaagaacagttaagtggttcatgtacagttgaagtagcagcatttaatgaatttacaaattttttatccgataaaactttagaacaagaatttgatttcattatatttgatacagctccaacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctagtcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatagatgaaattgaaaatagtaaagttcaatatttatttacaatgggaaaaggtggcgttggtaaaacgacagtagcaacgcaattagctacagtattatctaataaaggatatcgtgttcttttagcaactactgaccctactaaagaaattaatgttgaaaccacaagtaatttaaatactgcttatattgatgaagaacaagcattagaaaaatataaaaaagaagtactagcaacagtgaatgatgatacaccacaagacgatattgattatattatggaagatttaaaatcaccttgtacagaagaaatagcatttttcaaagcctttagtgacattatggagaatcaagaagacatggattacgtaattgtagatacagctcctacaggccataccttgctattacttgattctagtgaaaatcatcatagagaattaaagaaaaaatccactcaaactaccagtaatgttgaaacattattacccaaaattcaaaataaaaatttaacacagatgataatcgtaacattagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagtcttgataattactttgtcataccttatcgagtattagaatattga

SEQ ID NO: 76 - DNA, 1731 bp, Staphylococcus sp.; identical to SEQ ID NO: 75

SEQ ID NO: 77 - DNA, 1746 bp, Staphylococcus sp.
atggaggatgctgtggtggagatggatgctgttaagtacttaaataaattgaatttagataacgttgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgcttgctgagatgaaagaacagttaagtggttcatgtacagttgaagtagcagcatttaatgaatttacaaattttttatccgataaaactttagaacaagaatttgatttcattatatttgatacagctccaacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctagtcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatagatgaaattgaaaatagtaaagttcaatatttatttacaatgggaaaaggtggcgttggtaaaacgacagtagcaacgcaattagctacagtattatctaataaaggatatcgtgttcttttagcaactactgaccctactaaagaaattaatgttgaaaccacaagtaatttaaatactgcttatattgatgaagaacaagcattagaaaaatataaaaaagaagtactagcaacagtgaatgatgatacaccacaagacgatattgattatattatggaagatttaaaatcaccttgtacagaagaaatagcatttttcaaagcctttagtgacattatggagaatcaagaagacatggattacgtaattgtagatacagctcctacaggccataccttgctattacttgattctagtgaaaatcatcatagagaattaaagaaaaaatccactcaaactaccagtaatgttgaaacattattacccaaaattcaaaataaaaatttaacacagatgataatcgtaacattagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagtcttgataattactttgtcataccttatcgagtattagaatattga

SEQ ID NO: 78 - DNA, 1725 bp, Staphylococcus sp.
atggatgctgttaagtacttaaataaattgaatccagataacattgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgcttgctgagatgaaagaacagttaagtggttcatgtacagttgaagtagcagcatttaatgaatttacaaattttttatccgataaaactttagaacaagaatttgatttcattatatttgatacagctccaacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctagtcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatagatgaaattgaaaatagtaaagttcaatatttatttacaatgggaaaaggtggcgttggtaaaacgacagtagcaacgcaattagctacagtattatctaataaaggatatcgtgttcttttagcaactactgaccctactaaagaaattaatgttgaaaccacaagtaatttaaatactgcttatattgatgaagaacaagcattagaaaaatataaaaaagaagtactagcaacagtgaatgatgatacaccacaagacgatattgattatattatggaagatttaaaatcaccttgtacagaagaaatagcatttttcaaagcctttagtgacattatggagaatcaagaagacatggattacgtaattgtagatacagctcctacaggccataccttgctgttacttgattctagtgaaaatcatcatagagaattaaagaaaaaatcaactcaaactaccagtaatgttgaaacattattacccaaaattcaaaataaaaatttaacacagatgataattgtaacattagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagtcttgataattactttgtcataccttatcgagtattagaatattga

SEQ ID NO: 79 - DNA, 1725 bp, Staphylococcus sp.
atggatgctgttaagtacttaaataaattgaatttagataacgttgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgagggtattctaccagaagatgtgcttgctgagatgaaagaacagttaagtggttcatgtacagttgaagtagcagcatttaatgaatttacaaattttttatccgataaaactttagaacaagaatttgatttcattatatttgatacagctccaacaggtcacaccttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctagtcactcttctatatatgaaattcaaagagcacaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatagatgaaattgaaaatagtaaagttcaatatttatttacaatgggaaaaggtggcgttggtaaaacgacagtagcaacgcaattagctacagcattatctaataaaggatatcgtgttcttttagcaactactgaccctactaaagaaattaatgttgaaaccacaagtaatttaaatactgcttatattgatgaagaacaagcattagaaaaatataaaaaagaagtactagcaacagtgaatgatgatacaccacaagacgatattgattatattatggaagatttaaaatcaccttgtacagaagaaatagcatttttcaaagcctttagtgacattatggagaatcaagacgacatggattacgtaattgtagatacagctcctacaggccataccttgctgttacttgattctagtgaaaatcatcatagagaattaaagaaaaaatcaactcaaactaccagtaatgttgaaacattattacccaaaattcaaaataaaaatttaacacagatgataattgtaacattagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagtcttgataattactttgtcataccttatcgagtattagaatattga

SEQ ID NO: 80 - DNA, 1698 bp, Staphylococcus sp.
ttgaatttagataacgttgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaattttgacccgattgctgctgcagacgattataaagcacaatctatagaaccttatgaaggtattctaccagaagatgtgcttgctgagatgaaagaacagttaagtggttcatgtacagttgaagtagcagcatttaatgaatttacaaattttttatccgataaaactttagaacaagaatttgatttcattatatttgatacagctccaacaggtcataccttgagaatgcttgaattaccttctgcatggacagattatttaaatacaacgagtaatgacgcttcttgcttaggtcaattatcaggtttaaatgaaaatagagataaatataattcagcacttgaaaaactacgtaaccaagatgatacgaccatgatgttagttgcgagacctagtcactcttctatatatgaaattcaaagagcgcaacaagaattacaacaactgtcaatttctaaattcaaagtaatcattaacaactatatagaagaaagtcacggtttaatttcgagtcagatgaaatcagaacaagataaaaacattaatcattttactgaatggttaaataacaatcatgcttattacgttccatataaaaagcagaaagaagaaggtatagaaagtttaactaatctattaaatgatgataacttaattgaaaatgatgactttattgttgaagatcatccgcaattcaataaattaatagatgaaattgaaaatagtaaagttcaatatttatttacaatgggaaaaggtggcgttggtaaaacgacagtagcaacgcaattagctacagcattatctaataaaggatatcgtgttcttttagcaactactgaccctactaaagaaattaacgttgaaactacaagtaatttaaatactgcttatattgatgaagaacaagcattagaaaagtataaaaaagaagtactagccacagtgaatgatgatacaccacaagacgatattgattatattatggaagatttaaaatcaccttgtacagaagaaatagcatttttcaaagcctttagtgacattatggagaatcaagacgacatggattacgtaattgtagatacagctcctacaggccataccttgctgttacttgattctagtgaaaatcatcatagagaattaaagaaaaaatcaactcaaactaccagtaatgttgaaacattattacccaaaattcaaaataaaaatttaacacagatgataattgtaacattagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagtcttgataattactttgtcataccttatcgagtattagaatattga

SEQ ID NO: 81 - DNA, 1728 bp, Staphylococcus sp.
gtggaaatggatgctgttaaatacttaaataaattgaatttagataacattgagttaacaaaatatttgttttttactggtaaaggtggcgtaggcaaaacaacgatatcaagttttattgctttaaacttagcagagaatggaaagaaagtagctttagtaagtactgatccagctagtaatttacaagatgtatttcaaatggaattatctaataaattaactaaatatcaacctatacctaatctctctatagccaatttcgacccgattgttgctgcagacgattataaagcacaatctatagaaccttatgagggtattctaccagaagatgtgcttgctgaaatgaaagaacagttaagtggttcatgtacagttgaagtagcagcatttaatgaatttacaaattttttatccgataaaactttagaacaagaatttgatttcattatatttgatacagctcccacaggtcacactttgagaatgcttgaattaccttctgcatggacagattatttaaatacaacgagtaatgacgcttcttgcttaggtcaattatcaggtttaaatgaaaatagagttaaatataattcagcacttgaaaaactacgtaaccaagatgatacgaccatgatgttagttgcgagacctactcactcttctatatatgaaattcaaagagcgcaacaagaattacaacaactgtcaatttctaaattcaaagtaatcattaacaactatatagaagaaagtcacggtttaatttcgagtcagatgaaatcggaacaagataaaaacattaatcattttactgaatggttaaataacaatcatgcttattacgttccatataaaaatcagaaagaagaaggtatagaaaatttaactaatctattaaatgatgataacttaattgaaaatgatgactttattgttgaagatcatccgcaattcaataaattaatcgatgaaattgaaaatagtaaagttcaatatttatttacaatgggaaaaggtggcgttggtaaaacgacagtagcaacgcaattagctacaacgttatctaataaaggatatcgtgttcttttagcaactactgaccctactaaagaaattaatgttgaaactacaagtaatttaaatactgcttatattgatgaagaacaagcattagaaaagtataaaaaagaagtactagccacagtgaatgatgatacaccacaagacgatattgattatattatggaagatttaaaatcaccttgtacagaagaaatagcatttttcaaagcctttagtgacattatggagaatcaagacgacatggattacgtcattgtagatacagctcctacaggccataccttgctgttacttgattctagtgaaaatcatcatagagaattaaagaaaaaatcaactcaaactaccagtaatgttgaaacattattacctaaaattcaaaataaaaatttaacacagatgataatcgtaacactagcagaaaaaacaccttatttagaatctaaacgtttagtagaagatttaaatagagctaatataggccataattggtgggttgttaatcaatcgttagttacgctaaatcaacgtgatgacctttttagtaacaaaaaagaagatgaatcattttggataaacaagattaaaaatgaaagttttgacaattattttgtcataccttatggggggttatcataa

SEQ ID NO: 82 - DNA, 24 bp, Artificial Sequence
  Description of Artificial Sequence Synthetic primer
aaaatattcaaacagcagtacgta

SEQ ID NO: 83 - DNA, 30 bp, Artificial Sequence
  Description of Artificial Sequence Synthetic probe
atgagtgatttaaatgacctccaccattat

SEQ ID NO: 84 - DNA, 25 bp, Artificial Sequence
  Description of Artificial Sequence Synthetic primer
aaactcatctaaagaaccccattgt

SEQ ID NO: 85 - DNA, 25 bp, Artificial Sequence
  Description of Artificial Sequence Synthetic primer
tgcgaaagcatcacttgaattattg

SEQ ID NO: 86 - DNA, 27 bp, Artificial Sequence
  Description of Artificial Sequence Synthetic probe
ccaatccaacagcagcaacaacctgac

SEQ ID NO: 87 - DNA, 24 bp, Artificial Sequence
  Description of Artificial Sequence Synthetic primer
ctctacatgctgaaaagttctgtg

SEQ ID NO: 88 - DNA, 25 bp, Artificial Sequence
  Description of Artificial Sequence Synthetic primer
ctgacagaacaatcaataataaagg

SEQ ID NO: 89 - DNA, 35 bp, Artificial Sequence
  Description of Artificial Sequence Synthetic probe
agtaatcgaatatcatcgagtcatcgtatcttatg

SEQ ID NO: 90 - DNA, 25 bp, Artificial Sequence
  Description of Artificial Sequence Synthetic primer
cggaactaaattctatatcttcacc

SEQ ID NO: 91 - DNA, 25 bp, Artificial Sequence
  Description of Artificial Sequence Synthetic primer
cgatttagaggcacatatagcccaa

SEQ ID NO: 92 - DNA, 31 bp, Artificial Sequence
  Description of Artificial Sequence Synthetic probe
actttggtaatattcctcctcttcttctttt

SEQ ID NO: 93 - DNA, 25 bp, Artificial Sequence
  Description of Artificial Sequence Synthetic primer
tttagaggctcatacagaacattct

SEQ ID NO: 94 - DNA, 21 bp, Artificial Sequence
  Description of Artificial Sequence Synthetic primer
acctacatatgcatgtgtttt

SEQ ID NO: 95 - DNA, 35 bp, Artificial Sequence
  Description of Artificial Sequence Synthetic probe
acacacaaatgttgaattaaaagaacaccaaacaa

SEQ ID NO: 96 - DNA, 25 bp, Artificial Sequence
  Description of Artificial Sequence Synthetic primer
agtcaatttcctgttttagaaaatg

SEQ ID NO: 97 - DNA, 159 bp, Staphylococcus sp.
gatagtgttaagaaaattccttttgaaaatataaaatcagccgagtatgattctgacagaacaatcaataataaaggaaaagtaatcgaatatcatcgagtcatcgtatcttatggaaatggtgaagatatagaatttagttccgaacaatttgatagt

SEQ ID NO: 98 - DNA, 216 bp, Staphylococcus sp.
aagcatttaacgatttagaggcacatatagcccaaataagagccaaaaaagaagaagaggaggaatattaccaaagtttaaagaatgttctgtatgagcctctaaacaacgaatttgagtatgaatataaaaagcgaagtttattttcaaaagaaagtgagccaacaggacgagtcattttgaaagaagaagattataaaacattaaaagaacagg

SEQ ID NO: 99 - DNA, 219 bp, Staphylococcus sp.
aatttcttatttgaaaacaattttatcttcatcataagcatcaactgaataacctacatatgcatgtgtttttaatattaaatgttttggttcgcttttaaattcatattcataaatattaaatttgaattcttctgttattgtttggtgttcttttaattcaacatttgtgtgtaatgtaaatgtttgtaattcattttctaaaacaggaaattgact

SEQ ID NO: 100 - DNA, 219 bp, Staphylococcus sp.
aatttcttatttgaaaacaattttatcttcatcataagcatcaactgaataacctacatatgcatgtgtttttaatattaaatgttttggttcacttttaaattcatattcataaatattaaatttgaattcttctgttattgtgtggtgttcttttaattcaacatttgtgtgtagtgtaaatgtttgtaattcattttctaaaacaggaaattgact

SEQ ID NO: 101 - DNA, 29 bp, Artificial Sequence
  Description of Artificial Sequence Synthetic primer
gtggtaaatggtaatatcgacttaaaaca

SEQ ID NO: 102 - DNA, 35 bp, Artificial Sequence
  Description of Artificial Sequence Synthetic probe
acattttctttgctagagtagcactcgaattaggc

SEQ ID NO: 103 - DNA, 25 bp, Artificial Sequence
  Description of Artificial Sequence Synthetic primer
atttacgacttgttgcataccatca

SEQ ID NO: 104 - DNA, 27 bp, Artificial Sequence
  Description of Artificial Sequence Synthetic primer
gacttgttgcataccatcagttaatag

SEQ ID NO: 105 - DNA, 26 bp, Artificial Sequence
  Description of Artificial Sequence Synthetic primer
acttgttgcataccatcagttaatag

SEQ ID NO: 106 - DNA, 29 bp, Artificial Sequence
  Description of Artificial Sequence Synthetic primer
agtgttaactttagttgtagtttcaagtc

SEQ ID NO: 107 - DNA, 33 bp, Artificial Sequence
  Description of Artificial Sequence Synthetic probe
tagctcagcaaatgcatcacaaacagataacgg

SEQ ID NO: 108 - DNA, 30 bp, Artificial Sequence
  Description of Artificial Sequence Synthetic primer
cttgtgcttcactttttcttaaaagttgtt

SEQ ID NO: 109 - DNA, 34 bp, Artificial Sequence
  Description of Artificial Sequence Synthetic probe
agtagctcagcaaatgcatcacaaacagataacg

SEQ ID NO: 110 - DNA, 35 bp, Artificial Sequence
  Description of Artificial Sequence Synthetic probe
aagtagctcagcaaatgcatcacaaacagataacg

SEQ ID NO: 111 - DNA, 36 bp, Artificial Sequence
  Description of Artificial Sequence Synthetic probe
taagtagctcagcaaatgcatcacaaacagataacg

SEQ ID NO: 112 - DNA, 10 bp, Artificial Sequence
  Description of Artificial Sequence Synthetic probe
aaacacgtgc

SEQ ID NO: 113 - DNA, 10 bp, Artificial Sequence
  Description of Artificial Sequence Synthetic probe
ccttgttcca

SEQ ID NO: 114 - DNA, 10 bp, Artificial Sequence
  Description of Artificial Sequence Synthetic probe
cagggacgat

(19) United States
(12) Patent Application Publication
(10) Pub. No.: US 2011/0306510 A1
(43) Pub. Date: Dec. 15, 2011

(54) OPTIMIZED PPROBES AND PRIMERS AND METHODS OF USING SAME FOR THE DETECTION, SCREENING, ISOLATING AND SEQUENCING OF MRSA, MSSA STAPHYLOCOCCUS MARKERS, AND THE ANTIBIOTIC RESISTANCE GENE MEC A

(75) Inventors: Heinz R. Reiske, Framingham, MA (US); Chunyang Zheng, TianJin (CN); David L. Dolinger, Medway, MA (US); Alice A. Jacobs, Cambridge, MA (US); Philip T. Moen, Foxborough, MA (US); Chesley Leslin, Boston, MA (US); Juan Manuel Anzola, Waltham, MA (US); James R. Hully, Mundelein, IL (US)

(73) Assignee: Intelligent Medical Devices, Inc., Cambridge, MA (US)

(21) Appl. No.: 12/875,663

(22) Filed: Sep. 3, 2010

Related U.S. Application Data
(60) Provisional application No. 61/239,940, filed on Sep. 4, 2009.
(60) Provisional application No. 61/364,794, filed on Jul. 15, 2010.

(51) Int. Cl. C40B 30/04; G01N 33/53; C07H 21/04; C07H 21/00; C40B 40/06; C12Q 1/68; C12P 19/34

(52) U.S. Cl. 506/9; 435/6.12; 436/94; 435/91.5; 536/24.3; 506/16; 536/23.1; 536/24.33

(57) ABSTRACT

Described herein are primers and probes useful for the detection, screening, isolation and sequencing of MRSA, MSSA, Staphylococci markers, and the antibiotic resistance gene mecA.

### RELATED APPLICATION

This application claims the benefit of U.S. Provisional Application No. 61/239,940, filed on Sep. 4, 2009 and U.S. Provisional Application No. 61/364,794, filed on Jul. 15, 2010, the contents of which are incorporated by reference herein in their entirety.

### BACKGROUND

Staphylococcus is a genus of Gram-positive bacteria of the family Staphylococcaceae. Members of the genus Staphylococcus are widely distributed in nature, and some species have been shown to be important human pathogens, causing substantial rates of morbidity and mortality. Staphylococcus aureus, a coagulase-positive Staphylococcal species, is the main etiological agent and most frequently isolated microorganism from skin and soft tissue infections (SSTIs). Coagulase-negative Staphylococci (CNS or CoNS) have long been regarded as harmless skin commensals and dismissed as culture contaminants. However, the incidence of CoNS infection has increased with the increased use of prosthetic devices, central lines and other invasive technologies in hospital environments. Although Staphylococcus epidermidis accounts for the majority of CoNS infections, other species have also been identified in association with human infections. Multiresistant Staphylococcus sciuri, Staphylococcus haemolyticus and Staphylococcus hominis are also associated with skin and soft tissue infections. In addition, cases of multidrug resistant CoNS species in human infection have been reported. Limited treatment options and prolonged course of infection due to these CoNS species have been shown to have severe consequences for patients.

Staphylococcus infections are caused by Staphylococcus bacteria commonly found on the skin or in the nose of healthy individuals. In some circumstances, the bacteria invade the bloodstream, urinary tract, lungs or heart. Severe Staphylococcus infections usually occur in people who are already hospitalized or who have a chronic illness or weakened immune system, although otherwise healthy people can develop life-threatening Staphylococcus infections. Staphylococcus bacteria are very adaptable and some varieties have become resistant to one or more antibiotics. Up to half of the Staphylococcus bacteria found in hospitals today are resistant to methicillin, a β-lactam antibiotic. Methicillin-resistant Staphylococcus aureus (MRSA) is one of the major community-associated and health care-associated infections (HAI) responsible for increased morbidity, mortality, and prolonged hospitalization that results in increased costs of the whole healthcare system. Some Staphylococcus bacteria are vancomycin-resistant, and in rare cases, some isolates are resistant to both methicillin and vancomycin.

The genetic determinant of methicillin resistance (mec) has been localized to Staphylococcal cassette chromosome (SCC) elements harbored by S. aureus and some CoNS species. There is no allelic equivalent of mec in methicillin-susceptible S. aureus (MSSA). The mecA gene encodes an additional penicillin binding protein (PBP), PBP2a, which has a low affinity for all β-lactam antibiotics. mecA confers methicillin resistance in Staphylococcus and is located on a 21-67 kb mobile genetic element called SCCmec. SCCmec integrates into the orfX gene of S. aureus.

SCCmec elements are classified according to the particular combination of two parts: the recombinase complex and the mec complex. SCCmec types I-VII have been described, and SCC elements lacking mecA have also been reported. SCCmec IV has been regarded as the dominant SCCmec type in community-associated MRSA (CA-MRSA), while SCCmec I, II and III were prevalent in healthcare-associated MRSA strains (HA-MRSA). SCC is a conveyor not only of methicillin resistance and other antibiotic resistance genes, but also of virulence genes.

S. aureus produces numerous unique toxins and virulence factors, such as the toxic shock syndrome toxin (TSST-1), enterotoxins, and exotoxins, that can cause necrotizing pneumonia, complicated skin and soft tissue infections and septic shock. The genes seh and seo, which encode for superantigen enterotoxins H and O, have been found in close proximity to the SCCmec complex. Enterotoxins H and O have been reported in CA-MRSA. Enterotoxin H, encoded by two genes, lukS-PV and lukF-PV, is involved in acute toxic shock like syndromes. CA-MRSA can carry genes encoding toxins and virulence factors, such as Pantone-Valentine leukocidin (PVL) and lukE-lukD (leucocidin genes), which can cause severe skin and soft-tissue infections and necrotizing pneumonia. Virtually all CA-MRSA strains that cause soft tissue infections carry PVL and acquire PVL through bacteriophage encoding PVL genes.

The bacterial coagulase gene encodes the enzyme coagulase, which catalyzes the conversion of the protein fibrinogen to fibrin. Fibrin is subsequently involved in the clotting of blood. S. aureus is unique among most Staphylococcus species in that it harbors the coagulase gene—that is, S. aureus is coagulase-positive. Staphylococcus species lacking the coagulase gene are thus collectively referred to as coagulase-negative Staphylococcus (CNS or CoNS). Enzymatic assays for coagulase activity have been widely used in microbiological laboratories to differentiate S. aureus from other Staphylococcus species.

The nuc gene encodes the S. aureus thermostable nuclease. While other bacterial species possess genes encoding thermostable nucleases, the nuc gene sequence is unique to S. aureus and is frequently used as a species-specific marker for identifying S. aureus.

CoNS are a normal part of human microbial flora. For instance, S. epidermidis is a very common CoNS species found on human skin. While not usually pathogenic, CoNS species occasionally cause serious infections, some of which are resistant to antibiotics. S. epidermidis, among other CoNS species, can also harbor the SCCmec element and thus be resistant to methicillin. These methicillin-resistant CoNS (MR-CoNS) become resistant through the same mechanism as S. aureus, where the SCCmec integrates into the CoNS orfX ortholog. Methicillin-sensitive CoNS (MS-CoNS) are susceptible to methicillin.

The SCCmec element integrates into the S. aureus genome within the orfX gene. The exact function of the intact orfX gene is not known, but the gene itself is conserved among S. aureus strains. Furthermore, orthologs to orfX are found in other Staphylococcus species, suggesting that this gene has an essential function. The orfX gene itself is highly conserved within S. aureus, and is moderately conserved among different Staphylococcus species. The regions flanking the orfX gene however, are divergent among Staphylococcus species, but are highly conserved within S. aureus and MRSA.

Symptoms of Staphylococcus infections vary widely depending on the location and severity of the infection. Symptoms of MRSA include small red bumps, boils or reactions from spider bites, which turn into deep abscesses that require surgical draining. MRSA may remain confined to the skin, but can also cause infections in bones, joints, surgical wounds, the bloodstream, heart valves and lungs. Staphylococcus bacteria can be transmitted directly from person to person or indirectly through inanimate objects, such as pillowcases or towels. The bacteria can survive arid conditions, desiccation, temperature extremes, and high salt concentrations. Cooking will not destroy the toxins produced by Staphylococcus bacteria. A variety of factors can increase the risk of developing Staphylococcus infections, such as current or recent hospitalization, treatment with invasive devices, and participation in contact sports.

Prescribing treatments for S. aureus infection is difficult given the bacteria's potential for antibiotic resistance. Erythromycin resistance is the most common resistance marker in S. aureus; resistance to macrolides, clindamycin, and fluoroquinolones being found in many of the multidrug-resistant MRSA isolates.

Daptomycin, a lipopeptide antibiotic that is active against a wide variety of Gram-positive bacteria, has potent bactericidal activity. It is currently approved for the treatment of surgical site infections caused by Staphylococcus aureus. Daptomycin is also used for the treatment of a variety of vancomycin-resistant Enterococci (VRE) infections (as a result of E. faecalis and E. faecium), including soft tissue infections and bacteremia. However, there is an emergence of daptomycin-resistant strains such as S. aureus, S. epidermidis, and vancomycin-resistant E. faecium.

Vancomycin is often the antibiotic of choice to treat MRSA infections. While still an uncommon occurrence, vancomycin resistance has been documented in S. aureus.

Vancomycin-resistant Staphylococcus aureus (VRSA) becomes resistant to vancomycin through the acquisition of the van operon that is commonly found in vancomycin resistant Enterococcus (VRE). MRSA expression of both vancomycin and methicillin resistance is thought to be incompatible, as the penicillin binding protein (PBP) 2a (the protein encoded by the mecA gene) is incapable of cross-linking the peptidoglycan peptide modified by van genes. This presents a fitness cost to the host and in some cases this leads to deletion of the mecA gene by the van-harboring host. The SCCmec cassette that remains following mecA deletion is termed an “empty cassette.” Empty cassette S. aureus strains are often detected as MRSA by molecular tests amplifying the junction between the S. aureus genome and the SCCmec cassette, yielding a false positive test result for MRSA. Patients who develop VRSA infections usually have underlying health conditions, previous infections with MRSA, and recent hospitalizations. The spread of VRSA occurs through close physical contact with infected patients or contaminated material.

Vancomycin-intermediate Staphylococcus aureus (VISA) becomes resistant to vancomycin through a thickening of the cell wall, which is able to absorb the vancomycin and thus deplete the vancomycin that is available to reach cellular targets. The mechanism of VISA resistance is not known, but is believed to result from mutations in the S. aureus genome.

A rapid and accurate test panel for the screening or diagnosis of individuals for mecA gene variants, MRSA, MSSA and Staphylococcal markers would provide clinicians with an effective tool for identifying patients at risk for developing or who have developed methicillin resistance-associated diseases and subsequently supporting effective treatment regimens.

### SUMMARY

Described herein are nucleic acid probes and primers for screening, detecting, isolating and sequencing the mecA gene, the coagulase gene (coa), the thermostable nuclease gene (nuc) and orfX region (OXR) from the species Staphylococcus aureus, (S. aureus or SA) and marker genes for the coagulase-negative Staphylococci (CoNS specific markers) with a high degree of sensitivity and specificity. A screening test is critical to enable the quick and informative determination of whether or not an individual is colonized with MRSA, MSSA or both at the point of admission, or acquired through an individual's stay, in a hospital and/or medical care setting. Screening and surveillance of inanimate objects can eliminate the transmission of potentially deadly healthcare-associated infections (HAIs). A detection or diagnostic test that distinguishes multiple genes simultaneously is necessary because such detection is critical in patient and hospital personnel treatment.

Patient, personnel and inanimate object screening, combined with barrier isolation and contact precautions of MRSA or MSSA carriers, has been shown to be effective in controlling MRSA or MSSA infections; in some cases reducing to undetectable levels MRSA or MSSA in clinical facilities. The assays described herein are critical components of a resistance screening program to screen patients admitted to and personnel working in clinical settings for MRSA and/or MSSA. The assays described herein are also used to screen environmental surfaces for evidence that methicillin-resistant organisms are or were present in a hospital setting. Additionally, the assays described herein are used to identify or confirm that an isolate contains the methicillin resistance gene mecA.

Many facilities utilize culture-based methods for the determination, detection of and screening for antibiotic resistance, which require days to obtain the results. The methods of detection of and screening for the resistance markers described herein occurs within a minimal number of hours, allowing clinicians to rapidly determine the appropriate contact precautions or treatment for individuals harboring methicillin-resistant organisms, avoiding needless precautions for resistance-negative individuals and avoiding the careless use of antibiotics that will have little or no treatment efficacy.

In one embodiment, the present invention is directed to a method of detecting a methicillin-resistant S. aureus or methicillin-sensitive S. aureus in a biological sample, comprising the steps of: a) contacting a biological sample with a first oligonucleotide set designed to amplify and/or detect a S. aureus coa or nuc gene; a second oligonucleotide set designed to amplify and/or detect a S. aureus mecA gene; and a third oligonucleotide set designed to amplify and/or detect a S. aureus orfX region, wherein the third oligonucleotide set will not amplify or detect the orfX region if the orfX gene comprises an insertion sequence; and b) performing a nucleic acid amplification on the contacted sample, wherein amplification and/or detection of a product from both the first and second oligonucleotide sets and no amplification and/or no detection of a product from the third oligonucleotide set indicates the presence of methicillin-resistant S. aureus in the sample, and wherein amplification and/or detection of a product from both the first and third oligonucleotide sets and no amplification and/or no detection of a product from the second oligonucleotide set indicates the presence of methicillin-sensitive S. aureus in the sample. In a particular embodiment, the first oligonucleotide set is designed to amplify a S. aureus coa or nuc gene, and comprises one or more oligonucleotides comprising one or more sequences selected from the group consisting of SEQ ID NOS: 1, 3, 106 and 108. In a particular embodiment, the third oligonucleotide set is designed to amplify a S. aureus orfX region, and comprises one or more oligonucleotides comprising one or more sequences selected from the group consisting of SEQ ID NOS: 7, 9, 11, 13 and 15. In a particular embodiment, the second oligonucleotide set is designed to amplify a S. aureus mecA gene, and comprises one or more oligonucleotides comprising one or more sequences selected from the group consisting of SEQ ID NOS: 4, 6, 101 and 103-105. In a particular embodiment, the first oligonucleotide set comprises a probe that detects a S. aureus coa or nuc gene amplicon, wherein the probe comprises a sequence selected from the group consisting of: SEQ ID NOS: 2, 107 and 109-111. In a particular embodiment, the second oligonucleotide set comprises a probe that detects a S. aureus mecA gene amplicon, wherein the probe comprises a sequence selected from the group consisting of: SEQ ID NOS: 5 and 102. In a particular embodiment, third oligonucleotide set comprises a probe that detects a S. aureus orfX region amplicon, wherein the probe comprises a sequence selected from the group consisting of: SEQ ID NOS: 8, 10, 12, 14 and 16.

In one embodiment, the present invention is directed to a method of hybridizing one or more nucleic acid sequences comprising a sequence selected from the group consisting of: SEQ ID NOS: 1-28, 82-96, 101-111 to one or more target nucleic acids selected from the group consisting of: a methicillin resistance gene, an orfX region, a coa gene, a nuc gene, a CoNS specific marker gene, and combinations thereof, the method comprising contacting a sample comprising the target nucleic acid with the one or more nucleic acid sequences under conditions suitable for hybridization. In a particular embodiment, the methicillin resistance gene is mecA. In a particular embodiment, the target nucleic acid is contained in a nucleic acid selected from the group consisting of: a genomic sequence, a template sequence, a sequence derived from an artificial construct, genomic insert, cassette derived, inserted cassette, a plasmid, an insertion element, a naturally occurring plasmid and a naturally occurring transposable element. In a particular embodiment, the methods further comprise isolating the one or more hybridized target nucleic acids. In a particular embodiment, the methods further comprise quantitating the extent of hybridization of the one or more nucleic acids to the one or more target nucleic acids. In a particular embodiment, the methods further comprise sequencing the one or more hybridized target nucleic acids. In a particular embodiment, the methods further comprise monitoring and/or screening for the presence of the one or more hybridized target nucleic acids.

In one embodiment, the present invention is directed to a method of producing a nucleic acid product, comprising contacting one or more nucleic acid sequences selected from the group consisting of: SEQ ID NOS: 1, 3, 4, 6, 7, 9, 11, 13, 15, 17, 19, 20, 22-25, 27-29, 31, 36, 38, 40, 43, 45, 46, 48, 49, 51, 52, 56, 59, 60, 64-67, 69-72, 82, 84, 85, 87, 88, 90, 91, 93, 94, 96, 101, 103-106 and 108, to a template nucleic acid from a target selected from the group consisting of: a methicillin resistance gene, an orfX region, a coa gene, a nuc gene, a CoNS specific marker gene, a G. Stearothermophilus marker gene, and combinations thereof, under conditions suitable for nucleic acid polymerization. In a particular embodiment, the nucleic acid product is an amplicon produced using at least one forward primer selected from the group consisting of: SEQ ID NOS: 1 (coa); 4, 101 (mecA); 106 (nuc); 7 and 11 (orfX region); and 17, 20, 23-25, 82, 85, 88, 91 and 94 (CoNS specific marker); and at least one reverse primer selected from the group consisting of: SEQ ID NOS: 3 (coa); 6, 103, 104 and 105 (mecA); 108 (nuc); 9, 13 and 15 (orfX region); and 19, 22, 27, 28, 84, 87, 90, 93 and 96 (CoNS specific marker).

In a particular embodiment, the invention is directed to a probe or set of probes that hybridizes to the nucleic acid product of the methods described herein. In a particular embodiment, the probe or set of probes comprises one or more sequences selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker). In a particular embodiment, each probe sequence is labeled with a detectable label that is different from a detectable label associated with a different probe sequence. In a particular embodiment, the probe is labeled with a detectable label selected from the group consisting of: a fluorescent label, a chemiluminescent label, a quencher, a radioactive label, biotin and gold.

In one embodiment, the methods described herein for producing an amplicon further comprise contacting the one or more nucleic acids with one or more primers that produce an amplicon that is detectable by a process control probe. In a particular embodiment, a first probe comprises a sequence selected from the group consisting of: SEQ ID NOS: 2, 107, 109, 110 and 111, and a second probe comprises a sequence selected from the group consisting of: SEQ ID NOS: 5 and 102.

In one embodiment, the present invention is directed to a set of probes that hybridize to the amplicon(s) produced by the methods described herein, wherein a first probe comprises a sequence selected from the group consisting of: SEQ ID NOS: 2 (coa), 107, 109, 110 and 111 (nuc); a second probe comprises a selected from the group consisting of: SEQ ID NOS: 5 and 102 (mecA); and a third probe comprises a sequence selected from the group consisting of: SEQ ID NOS: 8, 10, 12, 14 and 16 (orfX region). In a particular embodiment, a first probe comprises a sequence selected from the group consisting of: SEQ ID NOS: 2 (coa), 107, 109, 110 and 111 (nuc); a second probe comprises a sequence selected from the group consisting of: SEQ ID NOS: 5 and 102 (mecA); and a third probe comprises a sequence selected from the group consisting of: SEQ ID NOS: 18, 21, 26, 83, 86, 89, 92 and 95 (CoNS marker). In a particular embodiment, a first probe comprises a sequence selected from the group consisting of: SEQ ID NOS: 2 (coa), 107, 109, 110 and 111 (nuc); a second probe comprises a sequence selected from the group consisting of: SEQ ID NOS: 5 and 102 (mecA); a third probe comprises a sequence selected from the group consisting of: SEQ ID NOS: 8, 10, 12, 14 and 16 (org region); and a fourth probe comprises a sequence selected from the group consisting of: SEQ ID NOS: 18, 21, 26, 83, 86, 89, 92 and 95 (CoNS marker). In a particular embodiment, the first probe is labeled with a first detectable label and the second probe is labeled with a second detectable label. In a particular embodiment, the first probe is labeled with a first detectable label, the second probe is labeled with a second detectable label, and the third probe is labeled with a third detectable label. In a particular embodiment, the first probe is labeled with a first detectable label, the second probe is labeled with a second detectable label, and the third probe is labeled with a third detectable label. In a particular embodiment, the first probe is labeled with a first detectable label, the second probe is labeled with a second detectable label, the third probe is labeled with a third detectable label, and the fourth probe is labeled with a fourth detectable label. In a particular embodiment, the detectable labels are selected from the group consisting of: a fluorescent label, a chemiluminescent label, a quencher, a radioactive label, biotin and gold.

In one embodiment, the present invention is directed to a method for detecting or screening for a methicillin resistance gene or an orfX region or a coa gene or a nuc gene or a CoNS specific marker gene in a sample, comprising: a) contacting the sample with at least one forward primer comprising a sequence selected from the group consisting of: SEQ ID NOS: 1 (coa); 4, 101 (mecA); 106 (nuc); 7 and 11 (orfX region); and 17, 20, 23, 24, 25, 82, 85, 88, 91 and 94 (CoNS specific marker); and at least one reverse primer selected from the group consisting of SEQ ID NOS: 3 (coa); 6, 103, 104 and 105 (mecA); 108 (nuc); 9, 13 and 15 (orfX region); and 19, 22, 27, 28, 84, 87, 90, 93 and 96 (CoNS specific marker) under conditions such that nucleic acid amplification occurs to yield an amplicon; and b) contacting the amplicon with one or more probes comprising one or more sequences selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker) under conditions such that hybridization of the probe to the amplicon occurs; wherein hybridization of the probe is indicative of a methicillin resistance gene or orfX region or coa gene or nuc gene or CoNS specific marker sequence in the sample. In a particular embodiment, each of the one or more probe sequences is labeled with a different detectable label. In a particular embodiment, the one or more probe sequences are labeled with the same detectable label. In a particular embodiment, the sample is selected from the group consisting of: blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, exudate, skin, gastric secretions, tears, oropharyngeal swabs, nasopharyngeal swabs, throat swabs, urine, anal-rectal swabs, feces, skin swabs, nasal aspirates, nasal wash, renal tissue and fluid therefrom, perfusion media, fluids and cells obtained by the perfusion of tissues of both human and animal origin, fluids and cells derived from the culturing of human cells, human stem cells, human cartilage, fibroblasts, pure cultures of bacterial fungal isolates, and swabs or washes of environmental surfaces, and other samples derived from environmental surfaces. In a particular embodiment, the sample is from a human. In a particular embodiment, the sample is non-human in origin. In a particular embodiment, the sample is derived from an inanimate object or environmental surface. In a particular embodiment, the at least one forward primer, the at least one reverse primer and the one or more probes are selected from the group consisting of: Groups 1-16, 74-80 of Table 4 and Groups 17-22, 69-73 of Table 5.

In one embodiment, the present invention is directed to a kit for detecting or screening for a methicillin resistance gene or orfX region or a coa gene or a nuc gene or a CoNS specific marker sequence in a sample, comprising one or more probe sequences comprising a sequence selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker). In a particular embodiment, the kit further comprises a) at least one forward primer comprising a sequence selected from the group consisting of: SEQ ID NOS: 1 (coa); 4, 101 (mecA); 106 (nuc); 7 and 11 (orfX); and 17, 20, 23, 24, 25, 82, 85, 88, 91 and 94 (CoNS specific marker); and b) at least one reverse primer comprising a sequence selected from the group consisting of: SEQ ID NOS: 3 (coa); 6, 103, 104 and 105 (mecA); 108 (nuc); 9, 13 and 15 (orfX); and 19, 22, 27, 28, 84, 87, 90, 93 and 96 (CoNS specific marker). In a particular embodiment, the kit further comprises reagents for quantitating, screening and/or sequencing a methicillin resistance gene or orfX region or a coa gene or a nuc gene or a CoNS specific marker sequence in the sample. In a particular embodiment, the one or more probe sequences are labeled with different detectable labels. In a particular embodiment, the one or more probe sequences are labeled with the same detectable label. In a particular embodiment, the kit further comprises an internal control and/or process control. In a particular embodiment, the at least one forward primer and the at least one reverse primer are selected from the group consisting of: Groups 1-16, 74-80 of Table 4, and Groups 17-22, 69-73 of Table 5.

In one embodiment, the present invention is directed to a method of diagnosing a condition, syndrome, colonization or disease in a human associated with a methicillin resistant organism or an orfX region or a coa gene or a nuc gene or a CoNS specific marker sequence comprising: a) contacting a sample with at least one forward and reverse primer set selected from the group consisting of: Groups 1-16, 74-80 of Table 4, and Groups 17-22, 69-73 of Table 5; b) conducting an nucleic acid amplification reaction, thereby producing an amplicon; and c) detecting the amplicon using one or more probes selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker), wherein the detection of an amplicon is indicative of the presence of a methicillin-resistant organism or an orfX region or a coa gene or a nuc gene or a CoNS specific marker sequence in the sample. In a particular embodiment, the sample is selected from the group consisting of: blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, exudate, skin, gastric secretions, tears, oropharyngeal swabs, nasopharyngeal swabs, throat swabs, urine, anal-rectal swabs, feces, skin swabs, nasal aspirates, nasal wash, renal tissue and fluid therefrom, perfusion media, fluids and cells obtained by the perfusion of tissues of both human and animal origin, fluids and cells derived from the culturing of human cells, human stem cells, human cartilage, fibroblasts, pure cultures of bacterial fungal isolates, and swabs or washes of environmental surfaces, and other samples derived from environmental surfaces. In a particular embodiment, the condition, syndrome or disease in a human associated with a methicillin-resistant organism is selected from the group consisting of: skin infection(s), boils, impetigo, cellulitis, scalded skin syndrome, food poisoning, abdominal cramps, nausea, vomiting, diarrhea, bacteremia, toxic shock syndrome, high fever, nausea, vomiting, rash on palms and soles, confusion, muscle aches, seizures, headache, septic arthritis, joint swelling, severe pain in the affected joint and shaking chills.

In one embodiment, the present invention is directed to a kit for binding, amplifying and sequencing a methicillin resistance gene or an orfX region or a coa gene or a nuc gene or a CoNS specific marker sequence in a sample, comprising: a) at least one forward primer comprising a sequence selected from the group consisting of: SEQ ID NOS: 1 (coa); 4, 101 (mecA); 106 (nuc); 7 and 11 (orfX); and 17, 20, 23, 24, 25, 82, 85, 88, 91 and 94 (CoNS specific marker); b) at least one reverse primer comprising a sequence selected from the group consisting of: SEQ ID NOS: 3 (coa); 6, 103, 104 and 105 (mecA); 108 (nuc); 9, 13 and 15 (orfX); and 19, 22, 27, 28, 84, 87, 90, 93 and 96 (CoNS specific marker); and c) reagents for the sequencing of amplified DNA fragments. In a particular embodiment, the kit further comprises reagents for quantitating, monitoring and/or screening a methicillin resistance gene or an orfX region or a coa gene or a nuc gene or a CoNS specific marker sequence in a sample. In a particular embodiment, the kit further comprises an internal control or process control primers and probes.

In one embodiment, the present invention is directed o a method of diagnosing a condition, syndrome or disease in a human associated with a methicillin resistance gene or an orfX region or a coa gene or a nuc gene or a CoNS specific marker sequence, comprising contacting a denatured target from a sample with one or more probe sequences comprising a sequence selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker) under conditions for hybridization to occur; wherein hybridization of the one or more probes to a denatured target is indicative of the presence of a methicillin resistance gene or an orfX region sequence or a coa gene or a nuc gene or a CoNS specific marker sequence in the sample. In a particular embodiment, the sample is selected from the group consisting of: blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, exudate, skin, gastric secretions, tears, oropharyngeal swabs, nasopharyngeal swabs, throat swabs, urine, anal-rectal swabs, feces, skin swabs, nasal aspirates, nasal wash, renal tissue and fluid therefrom, perfusion media, fluids and cells obtained by the perfusion of tissues of both human and animal origin, fluids and cells derived from the culturing of human cells, human stem cells, human cartilage, fibroblasts, pure cultures of bacterial fungal isolates, and swabs or washes of environmental surfaces, and other samples derived from environmental surfaces.

In one embodiment, the invention is directed to a probe that hybridizes to a methicillin—resistance gene target or an orfX region or a coa gene or a nuc gene or a CoNS specific marker sequence target. In a particular embodiment, the probe comprises a sequence selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92, 95 (CoNS specific marker). In a particular embodiment, the probe is labeled with a detectable label selected from the group consisting of: a fluorescent label, a chemiluminescent label, a quencher, a radioactive label, biotin and gold.

In one embodiment, the present invention is directed to a screening kit for binding, amplifying and sequencing a methicillin resistance gene or an orfX region or a coa gene or a nuc gene or a CoNS specific marker sequence in a sample, comprising: a) at least one forward primer comprising a sequence selected from the group consisting of: SEQ ID NOS: 1 (coa); 4, 101 (mecA); 106 (nuc); 7 and 11 (orfX); and 17, 20, 23, 24, 25, 82, 85, 88, 91 and 94 (CoNS specific marker); b) at least one reverse primer comprising a sequence selected from the group consisting of: SEQ ID NOS: 3 (coa); 6, 103, 104 and 105 (mecA); 108 (nuc); 9, 13 and 15 (orfX); and 19, 22, 27, 28, 84, 87, 90, 93 and 96 (CoNS specific marker); and c) reagents for the sequencing of amplified DNA fragments. In a particular embodiment, the screening kit further comprises an internal control and/or process control.

In one embodiment, the present invention is directed to a CoNS specific marker sequence comprising a sequence that is at least 70% homologous to a sequence selected from the group consisting of: SEQ ID NOS: 73-81 and 97-100.

In one embodiment, the present invention is directed to an isolated or synthesized nucleic acid sequence comprising a sequence selected from the group consisting of: SEQ ID NOS: 1-111.

In one embodiment, the present invention is directed to a primer set comprising at least one forward primer selected from the group consisting of: SEQ ID NOS: 1 (coa); 4, 101 (mecA); 106 (nuc); 7 and 11 (orfX region); and 17, 20, 23, 24, 25, 82, 85, 88, 91 and 94 (CoNS specific marker); and at least one reverse primer selected from the group consisting of: SEQ ID NOS: 3 (coa); 6, 103, 104 and 105 (mecA); 108 (nuc); 9, 13 and 15 (orfX region); and 19, 22, 27, 28, 84, 87, 90, 93 and 96 (CoNS specific marker). In a particular embodiment, the primer set is selected from the group consisting of: Groups 1-16, 74-80 of Table 4, Groups 17-22, 69-73 of Table 5, and Groups 23-68 of Table 6.

In one embodiment, the present invention is directed to a method of detecting a methicillin-coagulase-negative Staphyloccus in a biological sample, comprising the steps of: contacting a sample with (1) a first oligonucleotide set designed to amplify and/or detect a Staphylococcus) mecA gene; and (2) a second oligonucleotide set designed to amplify and/or detect a CoNS-marker gene, wherein amplification and/or detection of a product from both the first and second oligonucleotide sets indicates the presence of MR-CoNS in the sample.

The non-competitive internal control plasmid is a synthetic target that does not occur naturally in clinical sample types for which this assay is intended. The synthetic target sequence incorporates an artificial, random polynucleotide sequence with a known GC content. This internal control is detected by a forward primer, a reverse primer and a probe. A plasmid vector containing the internal control target sequence is included in the assay. The internal control plasmid is added directly to the reaction mix to monitor the integrity of the PCR reagents and the presence of PCR inhibitors.

The methicillin-resistant control plasmid contains partial sequences for one or more of the mecA targets. The positive control plasmid comprises forward primer, probe and reverse primer sequences for the given mecA gene targets. An artificial polynucleotide sequence is inserted within the positive control sequence corresponding to the given target to allow the amplicon generated by the target primer pairs to be differentiated from the amplicon derived by the same primer pairs from a natural target by size, by a unique restriction digest profile, and by a probe directed against the artificial sequence. The positive control plasmids are intended to be used as a control to confirm that the assay is performing within specifications.

Another embodiment of the invention is directed to a Gram-positive extraction/process control. Bacterial material from Geobacillus stearothermophilus, a Gram-positive bacteria not related to Staphylococcus, is incorporated into a kit (referred to hereinafter as the “extraction/process control bacterial material”). The extraction/process control can be used for monitoring the extraction process. The extraction/process control bacterial material will be cultured and aliquoted at a known titer. These aliquots will be provided as nucleic acid extraction controls. Known amounts of the process control bacterial material will be spiked into a test sample by the user of the test kit. Nucleic acids will be extracted from the test sample and subjected to PCR to detect Staphylococcus and the process control bacterial nucleic acids. Detection of the process control bacterial nucleic acids indicates that nucleic acid extraction from the test sample was successful. A listing of primers and probes used for the extraction/process control using Geobacillus stearothermophilus is provided in Table 6.

The oligonucleotides of the present invention and their resulting amplicons do not cross react and, thus, will work together without negatively impacting each other. The primers and probes of the present invention do not cross react with other potentially contaminating species that would be present in a sample matrix.

### BRIEF DESCRIPTION OF THE DRAWINGS

FIG. 1 is a schematic representation describing a strategy to detect MRSA, MSSA and/or MR-CoNS. 1a) S. aureus (SA) orfX gene region not detected in the presence of SCCmec as these primers would be 20-50 kb away from each other (this is too far for PCR; these primers do not detect other Staphylococcus species). 1b) SA orfX gene region is detected, which means SCCmec is not present, thus this cannot be MRSA; these primers would not detect other Staphylococcus species. 2) The mecA gene is detected, which means a methicillin-resistant organism or organisms are present. 3) The coa gene (which is located elsewhere in the S. aureus genome) is detected, which means S. aureus (either MRSA or MSSA) is present. Note: not drawn to scale.

FIG. 2 is a schematic representation describing a strategy to detect MRSA, MSSA and/or MR-CoNS. 1a) SA orfX gene region not detected in the presence of SCCmec as these primers would be 20-50 kb away from each other (this is too far for PCR; these primers do not detect other Staphylococcus species). 1b) SA orfX gene region is detected, which means SCCmec is not present, thus this cannot be MRSA; these primers would not detect other Staphylococcus species. 2) The mecA gene is detected, which means a methicillin-resistant organism or organisms are present. 3) The nuc gene (which is located elsewhere in the S. aureus genome) is detected, which means S. aureus (either MRSA or MSSA) is present. Note: not drawn to scale.

FIG. 3 is a plot demonstrating amplification of the intact orfX region from S. aureus and non-amplification and non-detection of the disrupted orfX region from MRSA using one group of orfX primers/probe of the invention.

FIG. 4 is a plot showing that one group of orfX primers/probe of the invention amplifies and specifically detects the S. aureus orfX region.

FIG. 5 is a plot demonstrating amplification of the mecA gene from MRSA using one group of mecA primers/probe of the invention.

FIG. 6 is a plot showing amplification of the mecA gene from MRSA using another group of mecA primers/probe of the invention.

FIG. 7 is a plot demonstrating amplification of the coa gene from S. aureus using the coa primers/probe group of the invention.

FIG. 8 is a plot showing that the coa primers/probe group of the invention amplifies and specifically detects the coa gene from S. aureus and MRSA.

FIG. 9 is a plot showing that one group of nuc primers/probe of the invention amplifies and specifically detects the nuc gene from MSSA and MRSA.

FIG. 10 is a plot showing that one group of CoNS (S. epidermidis) primers/probe amplifies and specifically detects the CoNS (S. epidermidis) marker.

### DETAILED DESCRIPTION

A diagnostic test that can detect multiple genes is necessary to prevent and treat HAIs. Methicillin resistant organisms are one of the major causative agents of HAIs. The primers and probes described herein can be used, for example, to screen patients for the presence of the mecA resistance gene and/or to confirm (detect) suspected cases of antibiotic resistance, e.g., in clinical isolates, including Staphylococcal pathogens, in a multiplex format. The present invention can screen individuals colonized with or detect patients infected with MRSA, MSSA and/or MR-CoNS using the described primers and probes.

Described herein are optimized probes and primers that, alone or in various combinations, allow for the amplification, detection, screening, isolation, and sequencing of the methicillin resistance gene mecA that can be found in clinical isolates, including Staphylococcal pathogens. Probes and primers that allow for the amplification, detection, screening, isolation and sequencing of the mecA gene in Staphylococcus, the S. aureus coagulase gene, the S. aureus thermostable nuclease gene, the presence of the orfX region or disruption of the orfX region, as well as CoNS specific markers, are included. Nucleic acid primers and probes for detecting bacterial genetic material, especially the resistance gene mecA, the S. aureus orfX region or disruption of the orfX region, the S. aureus coagulase and thermostable nuclease genes, and CoNS specific markers, and methods for designing and optimizing the respective primer and probe sequences, are described.

### Methicillin Resistance and Sensitivity; Staphylococci Markers

The mecA gene is found in various Staphylococcus organisms. mecA encodes a penicillin binding protein (PBP), PBP2a, which has a low affinity for all β-lactam antibiotics (Hanssen, A. & Ericson Sollid, J. (2006) FEMS Immunol Med Microbiol., 46:8-20). PBP2a is a high-molecular weight class, transpeptidase that catalyzes the formation of cross-bridges in the bacterial cell wall peptidoglycan. Assisted by the transglycosylase domain of the native PBP2 of S. aureus, it replaces PBPs involved in cell wall biosynthesis that have been inactivated by ligating β-lactams. Integration of the element is at a unique site called the bacterial chromosomal attachment site (attBSCC) downstream of a highly conserved open reading frame (ORF) of unknown function, designated orfX. SCCmec elements are classified according to the particular combination of two parts: the recombinase complex and the mec complex. SCCmec types I-VII have been described, and SCC elements lacking mecA have also been reported. (Kondo, Y. et al. (2007) Antimicrob Agents Chemother., 51:264-274; Berglund, C. et al. (2008) Antimicrob Agents Chemother., 52:3512-3516). SCCmec IV has been regarded as the dominant SCCmec type in community-associated MRSA (CA-MRSA), while SCCmec I, II and III are prevalent in healthcare-associated MRSA strains (HA-MRSA). SCC is a conveyor not only of methicillin resistance and other antibiotic resistance genes, but also of virulence genes. It has been speculated that SCCmec element is responsible for horizontal gene transfer between Staphylococci. It is important to note that there appears to be convergence between CA-MRSA and HA-MRSA that may lead to the two types becoming indistinguishable.

The mecA gene harbored on numerous SCCmec types—found in MRSA and MR-CoNS-is highly conserved. For this reason, amplification of the mecA gene is not suitable for differentiating MRSA from MR-CoNS. Therefore, a coagulase-negative Staphylococcus (CoNS)-specific marker has utility in distinguishing between MRSA and MR-CoNS in the presence of other S. aureus and methicillin resistance markers. The coagulase-negative (CoNS) markers are found in Staphylococcus hominis, Staphylococcus haemolyticus, and Staphylococcus epidermidis. Amplification of a CoNS marker and mecA in the absence of coa or nuc amplification indicates the presence of MR-CoNS Likewise, the amplification of coa or nuc and mecA in the absence of CoNS marker amplification indicates the presence of MRSA. The coagulase gene and thermostable nuclease gene represent excellent markers for S. aureus and can be amplified using specific PCR primers and probes.

The conserved elements within the orfX region are useful targets for the development of PCR primers and probes that can discriminate between S. aureus (MRSA or MSSA) and other Staphylococcus species. The orfX region, or a region associated with a different gene or genetic element, is a genetic space that is not limited to a particular open reading frame. It includes regulatory genetic elements as well as regions within a sufficiently close genetic proximity to regulate the orfX region (e.g., transcription of any of its coding regions), other gene or genetic element. In addition, a genetic region, e.g., the orfX region, includes sequences sufficiently proximal to the coding regions such that they can prime polymerase reactions across the reading frame(s) of, for example, the orfX region. A signal resulting from such PCR primers and probes targeting the orfX region is specifically indicative of S. aureus. Furthermore, when the orfX region primers and probes are combined with primers and probes directed to the coa gene, it becomes possible to discriminate between S. aureus and MRSA by virtue of the fact that the distance between the orfX region primer annealing sites increases from hundreds of bases to well over 20 kilobases upon integration of the SCCmec element into orfX. A distance of 20 kilobases or greater is too long a distance to efficiently amplify a target under the standard real-time PCR conditions commonly used in molecular diagnostic tests. Thus, the combination of (1) the absence of a signal from the orfX region primers and probes (i.e., an orfX region disruption)and (2) the presence of a signal from the coa or nuc primers and probes specifically indicates the presence of SCCmec. The presence of an SCCmec in S. aureus is interpreted as MRSA. If the S. aureus orfX gene region is detected, which means SCCmec is not present, then the sample cannot be MRSA.

The reagents and assays described herein can also be used to detect the presence of “empty cassette” strains as MSSA.

### Assays

Described herein are primers and probes to detect and/or screen for mecA, the S. aureus specific markers, coagulase and thermostable nuclease, the orfX region of S. aureus and specific markers for CoNS. The detection of and/or screening for these genes allows one to differentiate between MSSA, MRSA and coagulase-negative Staphylococci, e.g., S. epidermidis and S. haemolyticus, which may be methicillin-resistant (MR-CoNS or MR-CNS). MR-CoNS harbor the mecA gene, but do not have a coagulase gene. The coagulase gene is specific for S. aureus. The thermostable nuclease gene sequence is also specific for S. aureus and is used herein for identification of S. aureus.

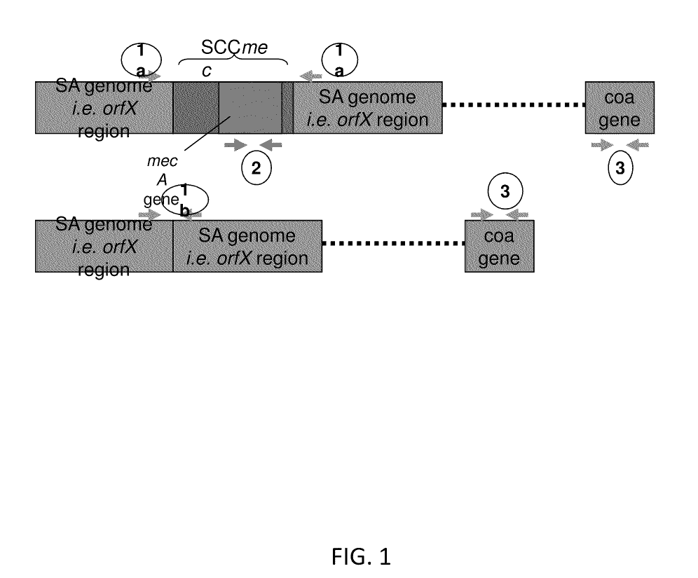

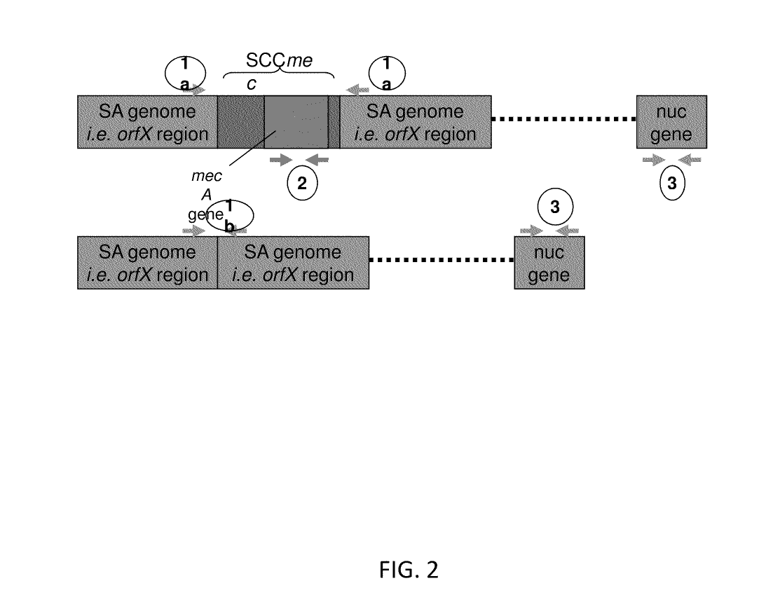

FIG. 1 illustrates the strategy encompassed by this invention for detection and/or screening of individuals for MRSA, MSSA and/or MR-CoNS. Tables 1 and 2 illustrate the possible results and interpretations for a test(s) involving primers and probes specific for the mecA, coa and nuc genes, the orfX region and the CoNS specific markers. The specific primers and probes for the detection of mecA, coa and the orfX region are described in Table 4. The specific primers and probes for the detection of CoNS are described in Table 5. FIG. 2 illustrates the same strategy using the nuc gene in place of the coa gene as an S. aureus specific marker.

A particular embodiment to detect or screen for MRSA may be divided into two modules. The first module may contain primers and probes directed to the following targets: (1) orfX region (specific for S. aureus); (2) coa or nuc (specific for S. aureus); and (3) mecA. The second module may contain primers and probes directed to the following targets: (1) orfX region (specific for coa or nuc positive Staphylococci species); (2) CoNS specific markers; and (3) mecA. Each of these modules may include a process control. Other embodiments include multiplexes in every various combination or singleplexes wherein each target is detected separately.

Tables 1 and 2 demonstrate possible diagnostic outcome scenarios using the probes and primers described herein in diagnostic and/or screening methods.

TABLE 1
Possible diagnostic or screening outcome scenarios using the probes and
primers of the present invention to detect MRSA and/or MSSA.

| | Target | | | |
|---|---|---|---|---|
| | | | | Gram |
| | | | | positive |
| | orfX | coa or | | extraction/ |
| Interpretation | region | nuc | mecA | process ctrl. |
| MSSA | + | + | − | + |
| MSSA | − | + | − | + |
| probable empty | | | | |
| cassette | | | | |
| MRSA | − | + | + | + |
| MSSA/MRSA | + | + | + | + |
| (1) MSSA/methicillin | +++ | +++ | + | + |
| resistant organism | | | | |
| (may or may not | | | | |
| include MRSA) | | | | |
| MSSA more | | | | |
| abundant | | | | |
| (2) MSSA/methicillin | + | + | +++ | + |
| resistant organism | | | | |
| (may or may not | | | | |
| include MRSA) | | | | |
| other methicillin | | | | |
| resistant organisms | | | | |
| more abundant | | | | |
| Methicillin | − | − | + | + |
| resistant organism, | | | | |
| S. aureus not | | | | |
| detected | | | | |
| Sample negative | − | − | − | + |
| Sample invalid | − | − | − | − |

+++ indicates quantity
(1) If the orfX region and the coa or nuc signals (i.e., Ct values) are comparable, then the presence of MSSA can be inferred. If these signals are greater than a signal corresponding to mecA, then the presence of MSSA and methicillin resistant organisms (may or may not include MRSA) can be inferred
(2) If the orfX region and the coa or nuc signals (i.e., Ct values) are comparable but lesser than the signal corresponding to mecA, then the presence of MSSA and methicillin resistant organisms (may or may not include MRSA) can also be inferred

TABLE 2
Possible diagnostic or screening outcome scenarios using the probes and primers of the
present invention to detect MRSA, MSSA and/or MR-CoNS.

| | Target | | | | |
|---|---|---|---|---|---|
| | | | | | Gram positive |
| | | | | CoNS | extraction/process |
| Interpretation | orfX region | coa or nuc | mecA | marker | ctrl. |
| MSSA | + | + | − | − | + |
| MSSA | − | + | − | − | + |
| probable empty | | | | | |
| cassette | | | | | |
| MRSA | − | + | + | − | + |
| MSSA/MRSA | + | + | + | − | + |
| MSSA/MR- | + | + | +++ | +++ | + |
| CoNS | | | | | |
| MR-CoNS more | | | | | |
| abundant | | | | | |
| (1) MSSA/MR- | +++ | +++ | + | + | |
| CoNS | | | | | |
| MSSA more | | | | | |
| abundant | | | | | |
| (2) MRSA/MR- | − | + | + | +++ | + |
| CoNS | | | | | |
| MR-CoNS more | | | | | |
| abundant | | | | | |
| MRSA/MR- | − | +++ | +++ | + | |
| CoNS | | | | | |
| MRSA more | | | | | |
| abundant | | | | | |
| MR-CoNS, | − | − | + | + | + |
| negative for S. | | | | | |
| aureus | | | | | |
| CoNS | − | − | − | + | + |
| Sample negative | − | − | − | − | + |
| Sample invalid | − | − | − | − | − |

+++ indicates quantity
(1) If the orfX region and the coa or nuc signals (i.e., Ct values) are comparable and the mecA and CoNS marker signals are comparable, the presence of MSSA and MR-CoNS can be inferred
(2) If the mecA and the coa or nuc signals are comparable and a signal is obtained for the CoNS marker, then the presence of MRSA and a CoNS (that may or may not be methicillin resistant) can be inferred

Detection of the process control indicates that the sample result is valid, where an absence of a signal corresponding to the process control indicates either an invalid result or that one or more of the specific targets are at a high starting concentration. A signal indicating a high starting concentration of specific target(s) in the absence of an internal control signal is considered to be a valid sample result.

The advantages of a multiplex format are: (1) simplified and improved testing and analysis; (2) increased efficiency and cost-effectiveness; (3) decreased turnaround time (increased speed of reporting results); (4) increased productivity (less equipment time needed); and (5) coordination/standardization of results for patients for multiple organisms (reduces error from inter-assay variation).

Screening and/or diagnosis/detection of the methicillin resistance gene can lead to earlier and more effective treatment of a subject. The methods for screening and/or detecting methicillin resistance described herein can be coupled with effective treatment therapies (e.g., antibiotics). The antibiotic classes comprising vancomycin and linezolid are often prescribed for treatment of a methicillin-resistant infection. The treatments for such infection will depend upon the clinical disease state of the patient, as determinable by one of skill in the art.

The present invention therefore provides a method for specifically screening and/or detecting for the presence of mecA and Staphylococci markers in a given sample using the primers and probes provided herein. The optimized primers and probes are useful, therefore, for identifying and diagnosing the causative or contributing agents of disease caused by MRSA, whereupon an appropriate treatment can then be administered to the individual to eradicate the bacteria.

The present invention provides one or more sets of primers that can anneal to the mecA gene, the S. aureus coa gene, the S. aureus nuc gene, the S. aureus orfX region, and CoNS specific markers genes and thereby amplify a target from a biological sample. The present invention provides, for example, at least a first primer and at least a second primer for the methicillin resistance gene, mecA; at least a first primer and at least a second primer for the S. aureus coa gene; at least a first primer and at least a second primer for the S. aureus nuc region; at least a first primer and at least a second primer for the S. aureus orfX region; at least a first primer and at least a second primer for a CoNS-specific marker; each of which comprises a nucleotide sequence designed according to the inventive principles disclosed herein, which are used together to amplify DNA from the methicillin resistance gene, S. aureus coagulase gene, the S. aureus thermostable nuclease gene, the S. aureus orfX region, and CoNS specific marker genes in a mixed-flora sample in a multiplex assay.

Also provided herein are probes that hybridize to mecA, the S. aureus coa gene, the S. aureus nuc gene, the S. aureus orfX region, and CoNS specific marker gene sequences and/or amplified products derived from the methicillin resistance gene, the S. aureus coagulase gene, the S. aureus thermostable nuclease gene, the S. aureus orfX region, and the CoNS specific marker gene sequences. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of DNA of the methicillin resistance gene mecA, S. aureus coa gene, S. aureus nuc gene, S. aureus orfX region, and CoNS specific marker genes because it permits the detection of bacterial-derived DNA at low template concentrations that might not be conducive to visual detection as a gel-stained amplification product.

Primers and probes are sequences that anneal to a bacterial genomic or bacterial genomic derived sequence, e.g., the antibiotic resistance gene mecA of Staphylococcus sequences (the “target” sequences). The target sequence can be, for example, an antibiotic resistance gene or a bacterial genome. In one embodiment, the entire gene sequence can be “scanned” for optimized primers and probes useful for detecting and screening for the genes of interest. In other embodiments, particular regions of the gene(s) can be scanned, e.g., regions that are, for example, documented in the literature or otherwise determined to be useful for detecting and screening for multiple genes, regions that are conserved, or regions where sufficient information is available in, for example, a public database, with respect to the antibiotic resistance genes.

Sets or groups of primers and probes are generated based on the target to be detected. The set of all possible primers and probes can include, for example, sequences that include the variability at every site based on the known antibiotic resistance genes, or the Staphylococci genes, or the primers and probes can be generated based on a consensus sequence of the target. The primers and probes are generated such that the primers and probes are able to anneal to a particular sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all known and characterized methicillin resistance and Staphylococci genes. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 by is Tm=81.5+0.41 (%(G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of antibiotic resistance and/or Staphylococci genes by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native target sequence. Even if a probe were originally generated based on a consensus, the validation of the probe is in its ability to specifically anneal and detect every, or a large majority of, target sequences. The particular scoring or ranking steps performed depend upon the intended use for the primer and/or probe, the particular target nucleic acid sequence, and the number of resistance genes of that target nucleic acid sequence. The methods of the invention provide optimal primer and probe sequences because they hybridize to all or a subset of a methicillin resistance gene, Staphyloccoccus genes and strains of the Staphylococci species. Once optimized, oligonucleotides are identified that can anneal to such genes, the sequences can then further be optimized for use, for example, in conjunction with another optimized sequence as a “primer set” or for use as a probe. A “primer set” is defined as at least one forward primer and one reverse primer.

Described herein are methods for using the primers and probes for producing a nucleic acid product, for example, comprising contacting one or more nucleic acid sequences of SEQ ID NOS: 1-28, 82-96, 101-111 to a sample comprising the methicillin resistance gene or S. aureus coagulase gene or S. aureus thermostable nuclease gene or S. aureus orfX region or CoNS-specific marker genes under conditions suitable for nucleic acid polymerization. The primers and probes can additionally be used to sequence the DNA of the methicillin resistance gene or S. aureus coagulase gene or S. aureus thermostable nuclease gene or S. aureus orfX region or CoNS-specific marker genes or used to, for example, detect and screen for the methicillin resistance gene and/or S. aureus coagulase gene and/or S. aureus thermostable nuclease gene and/or S. aureus orfX region and/or CoNS-specific marker genes in a clinical isolate sample, e.g., obtained from a subject, e.g., a mammalian subject. Particular combinations for amplifying DNA of methicillin resistance gene or S. aureus coagulase gene or S. aureus thermostable nuclease gene or S. aureus orfX region or CoNS-specific marker genes include, for example, using at least one forward primer selected from the group consisting of: SEQ ID NOS: 1 (coa); 4, 101 (mecA); 106 (nuc); 7 and 11 (orfX); and 17, 20, 23, 24, 25, 82, 85, 88, 91 and 94 (CoNS specific marker); and at least one reverse primer selected from the group consisting of SEQ ID NOS: 3 (coa); 6, 103, 104 and 105 (mecA); 108 (nuc); 9, 13 and 15 (orfX); and 19, 22, 27, 28, 84, 87, 90, 93 and 96 (CoNS specific marker).

Methods are described for detecting and/or screening for the methicillin resistance gene and/or Staphylococci genes in a sample, for example, comprising (1) contacting at least one forward and reverse primer set, e.g., SEQ ID NOS: 1 (coa); 4, 101 (mecA); 106 (nuc); 7 and 11 (orfX); and 17, 20, 23, 24, 25, 82, 85, 88, 91 and 94 (CoNS specific marker) (forward primers); and 3 (coa); 6, 103, 104 and 105 (mecA); 108 (nuc); 9, 13 and 15 (orfX); and 19, 22, 27, 28, 84, 87, 90, 93 and 96 (CoNS specific marker) (reverse primers) to a sample; (2) conducting an amplification; and (3) detecting the generation of an amplified product, wherein the generation of an amplified product indicates the presence of methicillin resistance from Staphylococcus pathogens or an S. aureus coagulase gene or an S. aureus thermostable nuclease gene or an S. aureus orfX region or CoNS specific markers in a clinical isolate sample.

The detection of amplicons using probes described herein can be performed, for example, using a labeled probe, e.g., the probe comprising a nucleotide sequence selected from the group consisting of: SEQ ID NOS: 2 (coa); 5, 102 (mecA); 107, 109, 110, 111 (nuc); 8, 10, 12, 14, 16 (org region); 18, 21, 26, 83, 86, 89, 92, 95 (CoNS specific marker) that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to dissociate the probe's reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

Alternative embodiments may utilize digital PCR.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

Primer or probe sequences can be ranked according to specific hybridization parameters or metrics that assign a score value indicating their ability to anneal to bacterial strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimmers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid sequences. The methods of determining the primers and probes also can comprise the step of allowing for one or more nucleotide changes when determining identity between the candidate primer and probe sequences and the target nucleic acid sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TaqMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid sequences are hybridized by an optimal forward primer and an optimal reverse primer, for example, by determining the number of base pair differences between target nucleic acid sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Tables 4-7. In addition, the sequences can be incorporated into longer sequences, provided they function to specifically anneal to and identify bacterial strains. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Tables 4-7 or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The probe and/or primer nucleic acid sequences of the invention are optimal for identifying numerous strains of a target nucleic acid, e.g., the methicillin-resistance gene and/or Staphylococci genes. In an embodiment, the nucleic acids of the invention are primers for the synthesis (e.g., amplification) of target nucleic acid sequences and/or probes for identification, isolation, detection, screening or analysis of target nucleic acid sequences, e.g., an amplified target nucleic acid that is amplified using the primers of the invention.

In other aspects, the invention also provides vectors (e.g., plasmid, phage, expression), cell lines (e.g., mammalian, insect, yeast, bacterial), and kits comprising any of the sequences of the invention described herein. The invention further provides known or previously unknown target nucleic acid strain sequences that are identified, for example, using the methods of the invention. In an embodiment, the target nucleic acid sequence is an amplification product. In another embodiment, the target nucleic acid sequence is a native or synthetic nucleic acid. The primers, probes, and target nucleic acid sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for screening for and/or detecting the presence of methicillin resistance (mecA) and/or Staphylococci genes (e.g., coa, nuc, orfX region and CoNS markers) in an individual and/or from a sample, are provided herein. An individual can be a human male, human female, human adult, human child, or human fetus. An individual can also be any mammal, reptile, avian, fish, or amphibian. Hence, an individual can be a primate, pig, horse, cattle, sheep, dog, rabbit, guinea pig, rodent, bird or fish. A sample includes any item, surface, material, clothing, or environment, for example, sewage or water treatment plants, in which it may be desirable to test for the presence of the methicillin resistance gene mecA and/or Staphylococci genes. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of methicillin resistance genes and Staphylococci genes.

A probe of the present invention can comprise a label such as, for example, a fluorescent label, a chemiluminescent label, a radioactive label, biotin, gold, dendrimers, aptamer, enzymes, proteins, quenchers and molecular motors. In an embodiment, the probe is a hydrolysis probe, such as, for example, a TaqMan® probe. In other embodiments, the probes of the invention are molecular beacons, any fluorescent probes, and probes that are replaced by any double stranded DNA binding dyes (e.g., SYBR Green® 1).

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of and screening for the mecA gene and/or Staphylococci genes (i.e., coa, OXR, Co-NS marker) can be performed by exposing such an oligonucleotide-covered surface to a sample such that the binding of a complementary strain DNA sequence to a surface-attached oligonucleotide elicits a detectable signal or reaction.

Oligonucleotides of the present invention also include primers for isolating and sequencing nucleic acid sequences derived from any identified or yet to be isolated and identified methicillin-resistance gene and/or Staphylococci genes.

One embodiment of the invention uses solid support-based oligonucleotide hybridization methods to detect and screen for gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence of the methicillin resistance gene and/or Staphylococci genes. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, Mass., USA) and ink-jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of genes (e.g., mecA, coa, nuc, orfX region and CoNS markers). Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting and screening for gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Table 4. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides, normalization controls, or expression controls. In one embodiment, the negative control is a mammalian gene that is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. A probe can be labeled with an enzyme or, alternatively, the probe is labeled with a moiety that is capable of binding to another moiety that is linked to the enzyme. For example, in the biotin-streptavidin interaction, the streptavidin is conjugated to an enzyme such as horseradish peroxidase (HRP). A chromogenic substrate is added to the reaction and is processed/cleaved by the enzyme. The product of the cleavage forms a color, either in the UV or visible spectrum. In another embodiment, streptavidin alkaline phosphatase can be used in a labeled streptavidin-biotin immunoenzymatic antigen detection system.

The invention also provides methods of labeling nucleic acid molecules with cleavable mass spectrometry tags (CMST; U.S. Patent Application No: 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect and screen for gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various Staphylococci genetic material or genetic material derived therefrom, or other genetic material derived therefrom, such as RNA, DNA, cDNA, or a PCR product.

A “sample” that is tested for the presence of mecA and Staphylococci genes (i.e., orfX region, coa, nuc, CoNS-specific markers) includes, but is not limited to a tissue sample, such as, for example, blood, serum, plasma, enriched peripheral blood mononuclear cells, neoplastic or other tissue obtained from biopsies, cerebrospinal fluid, saliva, fluids collected from the ear, eye, mouth, and respiratory airways, sputum, exudate, skin, tears, oropharyngeal swabs, nasopharyngeal swabs, throat swabs, urine, anal-rectal swabs, feces, skin swabs, nasal aspirates, nasal wash, fluids and cells obtained by the perfusion of tissues of both human and animal origin, and fluids and cells derived from the culturing of human cells, including human stem cells and human cartilage or fibroblasts. The tissue sample may be fresh, fixed, preserved, or frozen. A is sample also includes any item, surface, material, or clothing, or environment, for example, sewage or water treatment plants, in which it may be desirable to test for the presence of the methicillin resistance gene and Staphylococci genes. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of mecA and Staphylococci genes (i.e., org region, nuc, coa, CoNS-specific markers).

The target nucleic acid strain that is amplified may be RNA or DNA or a modification thereof. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g. fluorescent or radioactive) during an extension reaction. The detecting step comprises measuring fluorescence, mass, charge, and/or chemiluminescence.

The target nucleic acid strain may not need amplification and may be RNA or DNA or a modification thereof. If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking , and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus is sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there are an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 3 are three different 10-base candidate probe sequences that are targeted to different regions of a consensus target nucleic acid strain sequence. Each candidate probe sequence is compared to a total of 10 native sequences.

TABLE 3

| | | | | | | | | | | | |
|---|---|---|---|---|---|---|---|---|---|---|---|
| #1. | A | A | A | C | A | C | G | T | G | C | (SEQ ID NO: 82) |
| | 0.7 | 1.0 | 1.0 | 1.0 | 1.0 | 1.0 | 1.0 | 1.0 | 1.0 | 1.0 | |

→Number of target nucleic acid strain sequences that are
perfectly complimentary - 7. Three out of the ten sequences do
not have an A at position 1.

| | | | | | | | | | | | |
|---|---|---|---|---|---|---|---|---|---|---|---|
| #2. | C | C | T | T | G | T | T | C | C | A | (SEQ ID NO: 83) |
| | 1.0 | 0.9 | 1.0 | 0.9 | 0.9 | 1.0 | 1.0 | 1.0 | 1.0 | 1.0 | |

→Number of target nucleic acid strain sequences that are
perfectly complimentary - 7, 8, or 9. At least one target
nucleic acid strain does not have a C at position 2, T at
position 4, or G at position 5. These differences may all be
on one target nucleic acid strain molecule or may be on two or
three separate molecules.

| | | | | | | | | | | | |
|---|---|---|---|---|---|---|---|---|---|---|---|
| #3. | C | A | G | G | G | A | C | G | A | T | (SEQ ID NO: 84) |
| | 1.0 | 1.0 | 1.0 | 1.0 | 1.0 | 0.9 | 0.8 | 1.0 | 1.0 | 1.0 | |

→Number of target nucleic acid strain sequences that are
perfectly complimentary - 7 or 8. At least one target nucleic
acid strain does not have an A at position 6 and at least two
target nucleic acid strain do not have a C at position 7.
These differences may all be on one target nucleic acid
strain molecule or may be on two separate molecules.

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base-A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same nine target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid sequences are identified with perfect complementarity to all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complementary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TaqMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules is can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

(iv) Position (5′ to 3′) Of The Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan® probe. If there are two or more degenerate bases in a primer and probe set the ranking will initially select the sets where the degeneracies occur on different sequences.

### B. Coverage Score

The total number of aligned sequences is considered under a coverage score. A value is is assigned to each position based on how many times that position has been reported or sequenced. Alternatively, coverage can be defined as how representative the sequences are of the known strains, subtypes etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, bacterial strains and/or species that are linked to high frequencies of infection will have a higher score than strains that are generally regarded as benign. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain is identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted to BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical

Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to is determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting and/or Screening for the mecA, coa, nuc and the orfX Region Genes and CoNS Specific Markers

The set of primers and probes were then scored according to the methods described herein to identify the optimized primers and probes of Tables 4, 5 and 6. It should be noted that the primers can also be used as probes either in the presence or absence of amplification of a sample. The primers and probes directed to mecA, coa, nuc and the orfX region are identified in Table 4. The CoNS specific marker primers and probes are described in Table 5. The primers and probes for detecting Geobacillus stearothermophilus (Gram-Positive Process Control) are described in Table 6. Table 7 identifies the CoNS specific marker gene sequences.

TABLE 4
Optimized Primers and Probes for the Detection of and Screening for the S. aureus
Coagulase gene (coa), Thermostable nuclease gene (nuc), mecA Resistance Gene, and
orfX region (OXR)

| | | | |
|---|---|---|---|
| Group | | | |
| No. | Forward Primer | Probe | Reverse Primer |

coa

| | | | |
|---|---|---|---|
| 1 | SEQ ID NO: 1 | SEQ ID NO: 2 | SEQ ID NO: 3 |
| | GAAAGGTATTCAAGGAGAATCAAGT | AACCTCAAGCAACTGAAACAACAGAAGCATCACA | TTGTATTCACGGATACCTGTACCAG |

mecA

| | | | |
|---|---|---|---|
| 2 | SEQ ID NO: 4 | SEQ ID NO: 5 | SEQ ID NO: 6 |
| | TGAAATATTATTAGCTGATTCAGGTTACG | CCCAGTACAGATCCTTTCAATCTATAGCGCATT | CCAAACTTTGTTTTTCGTGTCTTTT |
| 74 | SEQ ID NO: 101 | SEQ ID NO: 102 | SEQ ID NO: 103 |
| | GTGGTAAATGGTAATATCGACTTAAAACA | ACATTTTCTTTGCTAGAGTAGCACTCGAATTAGGC | ATTTACGACTTGTTGCATACCATCA |
| 75 | SEQ ID NO: 101 | SEQ ID NO: 102 | SEQ ID NO: 104 |
| | GTGGTAAATGGTAATATCGACTTAAAACA | ACATTTTCTTTGCTAGAGTAGCACTCGAATTAGGC | GACTTGTTGCATACCATCAGTTAATAG |
| 76 | SEQ ID NO: 101 | SEQ ID NO: 102 | SEQ ID NO: 105 |
| | GTGGTAAATGGTAATATCGACTTAAAACA | ACATTTTCTTTGCTAGAGTAGCACTCGAATTAGGC | ACTTGTTGCATACCATCAGTTAATAG |

nuc

| | | | |
|---|---|---|---|
| 77 | SEQ ID NO: 106 | SEQ ID NO: 107 | SEQ ID NO: 108 |
| | AGTGTTAACTTTAGTTGTAGTTTCAAGTC | TAGCTCAGCAAATGCATCACAAACAGATAACGG | CTTGTGCTTCACTTTTTCTTAAAAGTTGTT |
| 78 | SEQ ID NO: 106 | SEQ ID NO: 109 | SEQ ID NO: 108 |
| | AGTGTTAACTTTAGTTGTAGTTTCAAGTC | AGTAGCTCAGCAAATGCATCACAAACAGATAACG | CTTGTGCTTCACTTTTTCTTAAAAGTTGTT |
| 79 | SEQ ID NO: 106 | SEQ ID NO: 110 | SEQ ID NO: 108 |
| | AGTGTTAACTTTAGTTGTAGTTTCAAGTC | AAGTAGCTCAGCAAATGCATCACAAACAGATAACG | CTTGTGCTTCACTTTTTCTTAAAAGTTGTT |
| 80 | SEQ ID NO: 106 | SEQ ID NO: 111 | SEQ ID NO: 108 |
| | AGTGTTAACTTTAGTTGTAGTTTCAAGTC | TAAGTAGCTCAGCAAATGCATCACAAACAGATAACG | CTTGTGCTTCACTTTTTCTTAAAAGTTGTT |

orfX region

| | | | |
|---|---|---|---|
| 3 | SEQ ID NO: 7 | SEQ ID NO: 8 | SEQ ID NO: 9 |
| | AAGTTAATAACTTGTGGATAACTGG | AATTCTGTATGAGGAGATAATAATTTGGAGGGTGT | CGTGGATTTAATGTCCACCATTT |
| 4 | SEQ ID NO: 7 | SEQ ID NO: 10 | SEQ ID NO: 9 |
| | AAGTTAATAACTTGTGGATAACTGG | AACACCCTCCAAATTATTATCTCCTCATACAGAAT | CGTGGATTTAATGTCCACCATTT |
| 5 | SEQ ID NO: 11 | SEQ ID NO: 8 | SEQ ID NO: 9 |
| | GTGTGAACAAGTTAATAACTTGTGG | AATTCTGTATGAGGAGATAATAATTTGGAGGGTGT | CGTGGATTTAATGTCCACCATTT |
| 6 | SEQ ID NO: 11 | SEQ ID NO: 10 | SEQ ID NO: 9 |
| | GTGTGAACAAGTTAATAACTTGTGG | AACACCCTCCAAATTATTATCTCCTCATACAGAAT | CGTGGATTTAATGTCCACCATTT |
| 7 | SEQ ID NO: 7 | SEQ ID NO: 12 | SEQ ID NO: 13 |
| | AAGTTAATAACTTGTGGATAACTGG | ATGAGGAGATAATAATTTGGAGGGTGTTAAATGGT | ATTGAATGAACGTGGATTTAATGTC |
| 8 | SEQ ID NO: 7 | SEQ ID NO: 8 | SEQ ID NO: 13 |
| | AAGTTAATAACTTGTGGATAACTGG | AATTCTGTATGAGGAGATAATAATTTGGAGGGTGT | ATTGAATGAACGTGGATTTAATGTC |
| 9 | SEQ ID NO: 7 | SEQ ID NO: 14 | SEQ ID NO: 15 |
| | AAGTTAATAACTTGTGGATAACTGG | TCCACCATTTAACACCCTCCAAATTATTATCTCCT | ATATTGAATGAACGTGGATTTAATG |
| 10 | SEQ ID NO: 7 | SEQ ID NO: 10 | SEQ ID NO: 13 |
| | AAGTTAATAACTTGTGGATAACTGG | AACACCCTCCAAATTATTATCTCCTCATACAGAAT | ATTGAATGAACGTGGATTTAATGTC |
| 11 | SEQ ID NO: 11 | SEQ ID NO: 12 | SEQ ID NO: 13 |
| | GTGTGAACAAGTTAATAACTTGTGG | ATGAGGAGATAATAATTTGGAGGGTGTTAAATGGT | ATTGAATGAACGTGGATTTAATGTC |
| 12 | SEQ ID NO: 11 | SEQ ID NO: 8 | SEQ ID NO: 13 |
| | GTGTGAACAAGTTAATAACTTGTGG | AATTCTGTATGAGGAGATAATAATTTGGAGGGTGT | ATTGAATGAACGTGGATTTAATGTC |
| 13 | SEQ ID NO: 11 | SEQ ID NO: 14 | SEQ ID NO: 15 |
| | GTGTGAACAAGTTAATAACTTGTGG | TCCACCATTTAACACCCTCCAAATTATTATCTCCT | ATATTGAATGAACGTGGATTTAATG |
| 14 | SEQ ID NO: 11 | SEQ ID NO: 10 | SEQ ID NO: 13 |
| | GTGTGAACAAGTTAATAACTTGTGG | AACACCCTCCAAATTATTATCTCCTCATACAGAAT | ATTGAATGAACGTGGATTTAATGTC |
| 15 | SEQ ID NO: 7 | SEQ ID NO: 16 | SEQ ID NO: 13 |
| | AAGTTAATAACTTGTGGATAACTGG | AGATAATAATTTGGAGGGTGTTAAATGGTG | ATTGAATGAACGTGGATTTAATGTC |
| 16 | SEQ ID NO: 11 | SEQ ID NO: 16 | SEQ ID NO: 13 |
| | GTGTGAACAAGTTAATAACTTGTGG | AGATAATAATTTGGAGGGTGTTAAATGGTG | ATTGAATGAACGTGGATTTAATGTC |

TABLE 5
Optimized Primers and Probes for the Detection of and Screening for the Primers and
Probes for Detecting Coagulase-negative Staphylococci (CoNS)

| | | | |
|---|---|---|---|
| Group | | | |
| No. | Forward Primer | Probe | Reverse Primer |

CoNS marker

| | | | |
|---|---|---|---|
| 17 | SEQ ID NO: 17 | SEQ ID NO: 18 | SEQ ID NO: 19 |
| | TTGTTGAAGATCATCCGCAATTCAA | ATTTATTTACAATGGGAAAAGGTGGCG | GTTGCTACTGTCGTTTTACCA |
| 18 | SEQ ID NO: 20 | SEQ ID NO: 21 | SEQ ID NO: 22 |
| | AAGCACAATCTATAGAACCTTATGA | ACTGTACATGAACCACTTAACTGTTCTTTCA | TGTAAATTCATTAAATGCTGCTACT |
| 19 | SEQ ID NO: 23 | SEQ ID NO: 18 | SEQ ID NO: 19 |
| | TGACTTTATTGTTGAAGATCATCCG | ATTTATTTACAATGGGAAAAGGTGGCG | GTTGCTACTGTCGTTTTACCA |
| 20 | SEQ ID NO: 24 | SEQ ID NO: 21 | SEQ ID NO: 22 |
| | TGCTGCAGACGATTATAAAGCACA | ACTGTACATGAACCACTTAACTGTTCTTTCA | TGTAAATTCATTAAATGCTGCTACT |
| 21 | SEQ ID NO: 25 | SEQ ID NO: 26 | SEQ ID NO: 27 |
| | ACCTAATCTCTCTATAGCCAATT | AGATTGTGCTTTATAATCGTCTGCAGCA | CATGAACCACTTAACTGTTCTTTCA |
| 22 | SEQ ID NO: 20 | SEQ ID NO: 21 | SEQ ID NO: 28 |
| | AAGCACAATCTATAGAACCTTATGA | ACTGTACATGAACCACTTAACTGTTCTTTCA | ATTCTTGTTCTAAAGTTTTATCGGA |
| 69 | SEQ ID NO: 82 | SEQ ID NO: 83 | SEQ ID NO: 84 |
| | AAAATATTCAAACAGCAGTACGTA | ATGAGTGATTTAAATGACCTCCACCATTAT | AAACTCATCTAAAGAACCCCATTGT |
| 70 | SEQ ID NO: 85 | SEQ ID NO: 86 | SEQ ID NO: 87 |
| | TGCGAAAGCATCACTTGAATTATTG | CCAATCCAACAGCAGCAACAACCTGAC | CTCTACATGCTGAAAAGTTCTGTG |
| 71 | SEQ ID NO: 88 | SEQ ID NO: 89 | SEQ ID NO: 90 |
| | CTGACAGAACAATCAATAATAAAGG | AGTAATCGAATATCATCGAGTCATCGTATCTTATG | CGGAACTAAATTCTATATCTTCACC |
| 72 | SEQ ID NO: 91 | SEQ ID NO: 92 | SEQ ID NO: 93 |
| | CGATTTAGAGGCACATATAGCCCAA | ACTTTGGTAATATTCCTCCTCTTCTTCTTTT | TTTAGAGGCTCATACAGAACATTCT |
| 73 | SEQ ID NO: 94 | SEQ ID NO: 95 | SEQ ID NO: 96 |
| | ACCTACATATGCATGTGTTTT | ACACACAAATGTTGAATTAAAAGAACACCAAACAA | AGTCAATTTCCTGTTTTAGAAAATG |

TABLE 6
Optimized Primers and Probes for Detecting Geobacillus stearothermophilus
(Gram-Positive Extraction/Process Control)

| Group | | | |
|---|---|---|---|
| No. | Forward Primer | Probe | Reverse Primer |
| 23 | SEQ ID NO: 29 | SEQ ID NO: 30 | SEQ ID NO: 31 |
| | GAAGGTTATTTGGCAGAAGAGAGC | TGCGGCCTCCACTTTTCGTCTTGTCTAG | GAAACGAAAGCGCTGTGTATCG |
| 24 | SEQ ID NO: 29 | SEQ ID NO: 32 | SEQ ID NO: 31 |
| | GAAGGTTATTTGGCAGAAGAGAGC | GCTGGAGCTAGACAAGACGAAAAGTGG | GAAACGAAAGCGCTGTGTATCG |
| 25 | SEQ ID NO: 29 | SEQ ID NO: 33 | SEQ ID NO: 31 |
| | GAAGGTTATTTGGCAGAAGAGAGC | CTCCACTTTTCGTCTTGTCTAGCTCCAG | GAAACGAAAGCGCTGTGTATCG |
| 26 | SEQ ID NO: 29 | SEQ ID NO: 34 | SEQ ID NO: 31 |
| | GAAGGTTATTTGGCAGAAGAGAGC | TTTCGTCTTGTCTAGCTCCAGCGCCTG | GAAACGAAAGCGCTGTGTATCG |
| 27 | SEQ ID NO: 29 | SEQ ID NO: 35 | SEQ ID NO: 31 |
| | GAAGGTTATTTGGCAGAAGAGAGC | AGACAAGACGAAAAGTGGAGGCCGCAATG | GAAACGAAAGCGCTGTGTATCG |
| 28 | SEQ ID NO: 36 | SEQ ID NO: 37 | SEQ ID NO: 38 |
| | TGGAGCTAGACAAGACGAAAAGTG | TGTCGATACACAGCGCTTTCGTTTCGTTCA | AACATGACTCGTTCCCTCAA |
| 29 | SEQ ID NO: 36 | SEQ ID NO: 39 | SEQ ID NO: 38 |
| | TGGAGCTAGACAAGACGAAAAGTG | TGAACGAAACGAAAGCGCTGTGTATCGACA | AACATGACTCGTTCCCTCAA |
| 30 | SEQ ID NO: 36 | SEQ ID NO: 37 | SEQ ID NO: 40 |
| | TGGAGCTAGACAAGACGAAAAGTG | TGTCGATACACAGCGCTTTCGTTTCGTTCA | TTTTGTCATTGAGAGAACATGACTC |
| 31 | SEQ ID NO: 36 | SEQ ID NO: 39 | SEQ ID NO: 40 |
| | TGGAGCTAGACAAGACGAAAAGTG | TGAACGAAACGAAAGCGCTGTGTATCGACA | TTTTGTCATTGAGAGAACATGACTC |
| 32 | SEQ ID NO: 36 | SEQ ID NO: 41 | SEQ ID NO: 40 |
| | TGGAGCTAGACAAGACGAAAAGTG | GGCCAACTGTCGATACACAGCGCTTTC | TTTTGTCATTGAGAGAACATGACTC |
| 33 | SEQ ID NO: 36 | SEQ ID NO: 42 | SEQ ID NO: 43 |
| | TGGAGCTAGACAAGACGAAAAGTG | TCAAAACTGAACGAAACGAAAGCGCTGTGTAT | GTCATTGAGAGAACATGACTCGTT |
| 34 | SEQ ID NO: 36 | SEQ ID NO: 44 | SEQ ID NO: 43 |
| | TGGAGCTAGACAAGACGAAAAGTG | ACACAGCGCTTTCGTTTCGTTCAGTTTTGAGG | GTCATTGAGAGAACATGACTCGTT |
| 35 | SEQ ID NO: 36 | SEQ ID NO: 37 | SEQ ID NO: 45 |
| | TGGAGCTAGACAAGACGAAAAGTG | TGTCGATACACAGCGCTTTCGTTTCGTTCA | TTGAGAGAACATGACTCGTTCCC |
| 36 | SEQ ID NO: 36 | SEQ ID NO: 39 | SEQ ID NO: 45 |
| | TGGAGCTAGACAAGACGAAAAGTG | TGAACGAAACGAAAGCGCTGTGTATCGACA | TTGAGAGAACATGACTCGTTCCC |
| 37 | SEQ ID NO: 36 | SEQ ID NO: 41 | SEQ ID NO: 45 |
| | TGGAGCTAGACAAGACGAAAAGTG | GGCCAACTGTCGATACACAGCGCTTTC | TTGAGAGAACATGACTCGTTCCC |
| 38 | SEQ ID NO: 46 | SEQ ID NO: 47 | SEQ ID NO: 48 |
| | GAGTCATGTTCTCTCAATGACAAAA | TTGAAAACTAGATAACCGGAAAAGCGGAGGC | CGAAGAACATTTGCCCATCG |
| 39 | SEQ ID NO: 49 | SEQ ID NO: 50 | SEQ ID NO: 51 |
| | GCGCTTTCGTTTCGTTCAG | TGAGGGAACGAGTCATGTTCTCTCAATGACAAAA | TCCGCTTTTCCGGTTATCTAGTTTT |
| 40 | SEQ ID NO: 52 | SEQ ID NO: 53 | SEQ ID NO: 48 |
| | TTTTGAGGGAACGAGTCATGT | CGGTTATCTAGTTTTCAAGGAACGATTTT | CGAAGAACATTTGCCCATCG |
| 41 | SEQ ID NO: 52 | SEQ ID NO: 54 | SEQ ID NO: 48 |
| | TTTTGAGGGAACGAGTCATGT | GCTTTTCCGGTTATCTAGTTTTCAAGGAACGATT | CGAAGAACATTTGCCCATCG |
| 42 | SEQ ID NO: 52 | SEQ ID NO: 55 | SEQ ID NO: 48 |
| | TTTTGAGGGAACGAGTCATGT | ATCGTTCCTTGAAAACTAGATAACCGGAAAAGCG | CGAAGAACATTTGCCCATCG |
| 43 | SEQ ID NO: 52 | SEQ ID NO: 47 | SEQ ID NO: 48 |
| | TTTTGAGGGAACGAGTCATGT | TTGAAAACTAGATAACCGGAAAAGCGGAGGC | CGAAGAACATTTGCCCATCG |
| 44 | SEQ ID NO: 56 | SEQ ID NO: 57 | SEQ ID NO: 51 |
| | GCCAACTGTCGATACACAGC | ATGACTCGTTCCCTCAAAACTGAACGAAACGAA | TCCGCTTTTCCGGTTATCTAGTTTT |
| 45 | SEQ ID NO: 56 | SEQ ID NO: 58 | SEQ ID NO: 51 |
| | GCCAACTGTCGATACACAGC | TTCGTTTCGTTCAGTTTTGAGGGAACGAGTCATG | TCCGCTTTTCCGGTTATCTAGTTTT |
| 46 | SEQ ID NO: 56 | SEQ ID NO: 57 | SEQ ID NO: 59 |
| | GCCAACTGTCGATACACAGC | ATGACTCGTTCCCTCAAAACTGAACGAAACGAA | GGTTATCTAGTTTTCAAGGAACGAT |
| 47 | SEQ ID NO: 56 | SEQ ID NO: 58 | SEQ ID NO: 59 |
| | GCCAACTGTCGATACACAGC | TTCGTTTCGTTCAGTTTTGAGGGAACGAGTCATG | GGTTATCTAGTTTTCAAGGAACGAT |
| 48 | SEQ ID NO: 60 | SEQ ID NO: 61 | SEQ ID NO: 51 |
| | CTGTCGATACACAGCGCTTTC | ACGAGTCATGTTCTCTCAATGACAAAA | TCCGCTTTTCCGGTTATCTAGTTTT |
| 49 | SEQ ID NO: 60 | SEQ ID NO: 62 | SEQ ID NO: 51 |
| | CTGTCGATACACAGCGCTTTC | GTCATTGAGAGAACATGACTCGTTCCCTCAAAAC | TCCGCTTTTCCGGTTATCTAGTTTT |
| 50 | SEQ ID NO: 60 | SEQ ID NO: 50 | SEQ ID NO: 51 |
| | CTGTCGATACACAGCGCTTTC | TGAGGGAACGAGTCATGTTCTCTCAATGACAAAA | TCCGCTTTTCCGGTTATCTAGTTTT |
| 51 | SEQ ID NO: 60 | SEQ ID NO: 63 | SEQ ID NO: 51 |
| | CTGTCGATACACAGCGCTTTC | TCGTTCAGTTTTGAGGGAACGAGTCATGTTCTC | TCCGCTTTTCCGGTTATCTAGTTTT |
| 52 | SEQ ID NO: 60 | SEQ ID NO: 61 | SEQ ID NO: 59 |
| | CTGTCGATACACAGCGCTTTC | ACGAGTCATGTTCTCTCAATGACAAAA | GGTTATCTAGTTTTCAAGGAACGAT |
| 53 | SEQ ID NO: 60 | SEQ ID NO: 62 | SEQ ID NO: 59 |
| | CTGTCGATACACAGCGCTTTC | GTCATTGAGAGAACATGACTCGTTCCCTCAAAAC | GGTTATCTAGTTTTCAAGGAACGAT |
| 54 | SEQ ID NO: 60 | SEQ ID NO: 50 | SEQ ID NO: 59 |
| | CTGTCGATACACAGCGCTTTC | TGAGGGAACGAGTCATGTTCTCTCAATGACAAAA | GGTTATCTAGTTTTCAAGGAACGAT |
| 55 | SEQ ID NO: 60 | SEQ ID NO: 63 | SEQ ID NO: 59 |
| | CTGTCGATACACAGCGCTTTC | TCGTTCAGTTTTGAGGGAACGAGTCATGTTCTC | GGTTATCTAGTTTTCAAGGAACGAT |
| 56 | SEQ ID NO: 64 | SEQ ID NO: 61 | SEQ ID NO: 51 |
| | TACACAGCGCTTTCGTTTCG | ACGAGTCATGTTCTCTCAATGACAAAA | TCCGCTTTTCCGGTTATCTAGTTTT |
| 57 | SEQ ID NO: 64 | SEQ ID NO: 62 | SEQ ID NO: 51 |
| | TACACAGCGCTTTCGTTTCG | GTCATTGAGAGAACATGACTCGTTCCCTCAAAAC | TCCGCTTTTCCGGTTATCTAGTTTT |
| 58 | SEQ ID NO: 64 | SEQ ID NO: 50 | SEQ ID NO: 51 |
| | TACACAGCGCTTTCGTTTCG | TGAGGGAACGAGTCATGTTCTCTCAATGACAAAA | TCCGCTTTTCCGGTTATCTAGTTTT |
| 59 | SEQ ID NO: 65 | SEQ ID NO: 53 | SEQ ID NO: 48 |
| | GGGAACGAGTCATGTTCTCTCAA | CGGTTATCTAGTTTTCAAGGAACGATTTT | CGAAGAACATTTGCCCATCG |
| 60 | SEQ ID NO: 65 | SEQ ID NO: 54 | SEQ ID NO: 48 |
| | GGGAACGAGTCATGTTCTCTCAA | GCTTTTCCGGTTATCTAGTTTTCAAGGAACGATT | CGAAGAACATTTGCCCATCG |
| 61 | SEQ ID NO: 65 | SEQ ID NO: 55 | SEQ ID NO: 48 |
| | GGGAACGAGTCATGTTCTCTCAA | ATCGTTCCTTGAAAACTAGATAACCGGAAAAGCG | CGAAGAACATTTGCCCATCG |
| 62 | SEQ ID NO: 65 | SEQ ID NO: 47 | SEQ ID NO: 48 |
| | GGGAACGAGTCATGTTCTCTCAA | TTGAAAACTAGATAACCGGAAAAGCGGAGGC | CGAAGAACATTTGCCCATCG |
| 63 | SEQ ID NO: 66 | SEQ ID NO: 61 | SEQ ID NO: 51 |
| | CGCTTTCGTTTCGTTCAGTTTTGA | ACGAGTCATGTTCTCTCAATGACAAAA | TCCGCTTTTCCGGTTATCTAGTTTT |
| 64 | SEQ ID NO: 67 | SEQ ID NO: 68 | SEQ ID NO: 69 |
| | GGCTCTCGAAGCCAAATGTTC | GGCTCTCTTCTGCCAAATAACCTTCCCC | TCCACTTTTCGTCTTGTCTAGCTC |
| 65 | SEQ ID NO: 70 | SEQ ID NO: 68 | SEQ ID NO: 69 |
| | CGAAGCCAAATGTTCTTCACC | GGCTCTCTTCTGCCAAATAACCTTCCCC | TCCACTTTTCGTCTTGTCTAGCTC |
| 66 | SEQ ID NO: 71 | SEQ ID NO: 53 | SEQ ID NO: 48 |
| | AGTCATGTTCTCTCAATGACA | CGGTTATCTAGTTTTCAAGGAACGATTTT | CGAAGAACATTTGCCCATCG |
| 67 | SEQ ID NO: 72 | SEQ ID NO: 54 | SEQ ID NO: 48 |
| | AGTCATGTTCTCTCAATGACAA | GCTTTTCCGGTTATCTAGTTTTCAAGGAACGATT | CGAAGAACATTTGCCCATCG |
| 68 | SEQ ID NO: 72 | SEQ ID NO: 55 | SEQ ID NO: 48 |
| | AGTCATGTTCTCTCAATGACAA | ATCGTTCCTTGAAAACTAGATAACCGGAAAAGCG | CGAAGAACATTTGCCCATCG |

TABLE 7
CoNS specific marker gene sequences

| SEQ ID NO: | CoNS Specific Marker Sequence |
|---|---|
| SEQ ID NO: 73 | ATGGAGATGGATGCTGTTAAATATTTAAATAAATTGAATCTAGATAACATTGAGTTAACAAAATATTTGTTTTTTACTGGTAAAGGT |
| | GGCGTAGGCAAAACGACGATATCAAGTTTTATTGCTTTAAACTTAGCAGAGAATGGAAAGAAAGTAGCTTTAGTAAGTACTGATCC |
| | AGCTAGTAATTTACAAGATGTATTTCAAATGGAATTATCTAATAAATTAACTAAATATCAACCTATACCTAATCTCTCTATAGCCAA |
| | TTTTGACCCGATTGCTGCTGCAGACGATTATAAAGCACAATCTATAGAACCTTATGAGGGTATTCTACCAGAAGATGTGCTTTCTGA |
| | AATGAAAGAACAGTTAAGTGGTTCATGTACAGTTGAAGTAGCAGCATTTAATGAATTTACAAATTTTTTATCCGATAAAACTTTAGA |
| | ACAAGAATTTGATTTCATTATATTTGATACAGCTCCAACAGGTCACACCTTGAGAATGCTTGAATTACCTTCTGCATGGACAGATTA |
| | TTTAAATACAACGAGTAATGACGCTTCTTGCTTAGGTCAATTATCAGGTTTAAATGAAAATAGAGTTAAATATAATTCAGCACTTGA |
| | AAAACTACGTAACCAAGATGATACGACCATGATGTTAGTTGCGAGACCTACTCACTCTTCTATATATGAAATTCAAAGAGCGCAAC |
| | AAGAATTACAACAACTGTCAATTTCTAAATTCAAAGTAATCATTAACAACTATATAGAAGAAAGTCACGGTTTAATTTCGAGTCAG |
| | ATGAAATCAGAACAAGATAAAAACATTAATCATTTTACTGAATGGTTAAATAACAATCATGCTTATTACGTTCCATATAAAAAGCA |
| | GAAAGAAGAAGGTATAGAAAGTTTAACTAATCTATTAAATGATGATAACTTAATTGAAAATGATGACTTTATTGTTGAAGATCATC |
| | CGCAATTCAATAAATTAATCGATGAAATTGAAAATAGTAAAGTTCAATATTTATTTACAATGGGAAAAGGTGGCGTTGGTAAAACG |
| | ACAGTAGCAACGCAATTAGCTACAACGTTATCTAATAAAGGATATCGTGTTCTTTTAGCAACTACTGACCCTACTAAAGAAATTAAT |
| | GTTGAAACTACAAGTAATTTAAATACTGCTTATATTGATGAAGAACAAGCATTAGAAAAATATAAAAAAGAAGTACTAGCCACAGT |
| | GAATGATGATACACCACAAGACGATATTGATTATATTATGGAAGATTTAAAATCACCTTGTACAGAAGAAATAGCATTTTTCAAAG |
| | CCTTTAGTGACATTATGGAGAATCAAGACGACGTGGATTACGTCATTGTAGATACAGCTCCAACAGGCCATACCTTGCTGTTACTTG |
| | ATTCTAGTGAAAATCATCATAGAGAATTAAAGAAAAAATCAACTCAAACTACCAGTAATGTTGAAACATTATTACCTAAGATTCAA |
| | AATAAAAATTTAACACAGATGATAATCGTAACACTAGCAGAAAAAACACCTTATTTAGAATCTAAACGTTTAGTAGAAGATTTAAA |
| | TAGAGCTAATATAGGCCATAATTGGTGGGTTGTTAATCAATCGTTAGTTACACTAAATCAACGTGATGACCTTTTTAGTAACAAAAA |
| | AGAAGATGAATCATTTTGGATAAACAAGATTAAAAATGAAAGTCTTAATAATTACTTTGTCATACCTTATCGAATATCAGAATATTA |
| | A |
| SEQ ID NO: 74 | GTGGAGATGGATGCTGTTAAATACTTAAATAAATTGAACCTAAATAACATTGAGTTAACAAAATATTTGTTTTTTACTGGTAAAGGT |
| | GGCGTAGGCAAAACAACGATATCAAGTTTTATTGCTTTAAACTTAGCAGAGAATGGAAAGAAAGTAGCTTTAGTAAGTACTGATCC |
| | AGCTAGTAATTTACAAGATGTATTTCAAATGGAATTATCTAATAAATTAACTAAATATCAACCTATACCTAATCTCTCTATAGCCAA |
| | TTTTGACCCGATTGCTGCTGCAGACGATTATAAAGCACAATCTATAGAACCTTATGAGGGTATTCTACCAGAAGATGTGCTTTCTGA |
| | AATGAAAGAACAGTTAAGTGGTTCATGTACAGTTGAAGTAGCAGCATTTAATGAATTTACAAATTTTTTATCCGATAAAACTTTAGA |
| | ACAAGAATTTGATTTCATTATATTTGATACAGCTCCCACAGGTCACACCTTGAGAATGCTTGAATTACCTTCTGCATGGACAGATTA |
| | TTTAAATACAACGAGTAATGACGCTTCTTGCTTAGGTCAATTATCAGGTTTAAATGAAAATAGAGTTAAATATAATTCAGCACTTGA |
| | AAAACTACGTAACCAAGATGATACGACCATGATGTTAGTTGCGAGACCTACTCACTCTTCTATATATGAAATTCAAAGAGCGCAAC |
| | AAGAATTACAACAACTGTCAATTTCTAAATTCAAAGTAATCATTAACAACTATATAGAAGAAAGTCACGGTTTAATTTCGAGTCAG |
| | ATGAAATCAGAACAAGATAAAAACATTAATCATTTTACTGAATGGTTAAATAACAATCATGCTTATTACGTTCCATATAAAAAGCA |
| | GAAAGAAGAAGGTATAGAAAGTTTAACTAATCTATTAAATGATGATAACTTAATTGAAAATGATGACTTTATTGTTGAAGATCATC |
| | CGCAATTCAATAAATTAATCGATGAAATTGAAAATAGTAAAGTTCAATATTTATTTACAATGGGAAAAGGTGGCGTTGGTAAAACG |
| | ACAGTAGCAACGCAATTAGCTACAACGTTATCTAATAAAGGATATCGTGTTCTTTTAGCAACTACTGACCCTACTAAAGAAATTAAT |
| | GTTGAAACCACAAGTAATTTAAATACTGCTTATATTGATGAAGAACAAGCATTAGAAAAGTATAAAAAAGAAGTACTAGCCACAGT |
| | GAATGATGATACACCACAAGACGATATCGATTATATTATGGAAGATTTAAAATCACCTTGTACAGAAGAAATAGCATTTTTCAAAG |
| | CCTTTAGTGACATTATGGAGAATCAAGACGACATGGATTACGTCATTGTAGATACAGCTCCTACAGGCCATACTTTGCTTTTACTTG |
| | ATTCTAGTGAAAATCATCATAGAGAATTAAAGAAAAAATCAACTCAAACTACCAGTAATGTTGAAACATTATTACCTAAGATTCAA |
| | AATAAAAATTTAACACAGATGATAATCGTAACACTAGCAGAAAAAACACCTTATTTAGAATCTAAACGTTTAGTAGAAGATTTAAA |
| | TAGAGCTAATATAGGTCATAATTGGTGGGTCGTTAATCAATCGTTAGTTACGCTAAATCAACGTGATGACCTTTTTAGTAACAAAAA |
| | AGAAGATGAATCATTTTGGATAAACAAAATTAAAAATGAAAGTTTTGATAATTACTTTGTCATACCTTATCGAATATCAGAATGTTA |
| | A |
| SEQ ID NO: 75 | GTGGAGATGGATGCTGTTAAGTACTTAAATAAATTGAATTTAGATAACGTTGAGTTAACAAAATATTTGTTTTTTACTGGTAAAGGT |
| | GGCGTAGGCAAAACAACGATATCAAGTTTTATTGCTTTAAACTTAGCAGAGAATGGAAAGAAAGTAGCTTTAGTAAGTACTGATCC |
| | AGCTAGTAATTTACAAGATGTATTTCAAATGGAATTATCTAATAAATTAACTAAATATCAACCTATACCTAATCTCTCTATAGCCAA |
| | TTTTGACCCGATTGCTGCTGCAGACGATTATAAAGCACAATCTATAGAACCTTATGAGGGTATTCTACCAGAAGATGTGCTTGCTGA |
| | GATGAAAGAACAGTTAAGTGGTTCATGTACAGTTGAAGTAGCAGCATTTAATGAATTTACAAATTTTTTATCCGATAAAACTTTAGA |
| | ACAAGAATTTGATTTCATTATATTTGATACAGCTCCAACAGGTCACACCTTGAGAATGCTTGAATTACCTTCTGCATGGACAGATTA |
| | TTTAAATACAACGAGTAATGACGCTTCTTGCTTAGGTCAATTATCAGGTTTAAATGAAAATAGAGTTAAATATAATTCAGCACTTGA |
| | AAAACTACGTAACCAAGATGATACGACCATGATGTTAGTTGCGAGACCTAGTCACTCTTCTATATATGAAATTCAAAGAGCGCAAC |
| | AAGAATTACAACAACTGTCAATTTCTAAATTCAAAGTAATCATTAACAACTATATAGAAGAAAGTCACGGTTTAATTTCGAGTCAG |
| | ATGAAATCAGAACAAGATAAAAACATTAATCATTTTACTGAATGGTTAAATAACAATCATGCTTATTACGTTCCATATAAAAAGCA |
| | GAAAGAAGAAGGTATAGAAAGTTTAACTAATCTATTAAATGATGATAACTTAATTGAAAATGATGACTTTATTGTTGAAGATCATC |
| | CGCAATTCAATAAATTAATAGATGAAATTGAAAATAGTAAAGTTCAATATTTATTTACAATGGGAAAAGGTGGCGTTGGTAAAACG |
| | ACAGTAGCAACGCAATTAGCTACAGTATTATCTAATAAAGGATATCGTGTTCTTTTAGCAACTACTGACCCTACTAAAGAAATTAAT |
| | GTTGAAACCACAAGTAATTTAAATACTGCTTATATTGATGAAGAACAAGCATTAGAAAAATATAAAAAAGAAGTACTAGCAACAGT |
| | GAATGATGATACACCACAAGACGATATTGATTATATTATGGAAGATTTAAAATCACCTTGTACAGAAGAAATAGCATTTTTCAAAG |
| | CCTTTAGTGACATTATGGAGAATCAAGAAGACATGGATTACGTAATTGTAGATACAGCTCCTACAGGCCATACCTTGCTATTACTTG |
| | ATTCTAGTGAAAATCATCATAGAGAATTAAAGAAAAAATCCACTCAAACTACCAGTAATGTTGAAACATTATTACCCAAAATTCAA |
| | AATAAAAATTTAACACAGATGATAATCGTAACATTAGCAGAAAAAACACCTTATTTAGAATCTAAACGTTTAGTAGAAGATTTAAA |
| | TAGAGCTAATATAGGCCATAATTGGTGGGTTGTTAATCAATCGTTAGTTACGCTAAATCAACGTGATGACCTTTTTAGTAACAAAAA |
| | AGAAGATGAATCATTTTGGATAAACAAGATTAAAAATGAAAGTCTTGATAATTACTTTGTCATACCTTATCGAGTATTAGAATATTG |
| | A |
| SEQ ID NO: 76 | GTGGAGATGGATGCTGTTAAGTACTTAAATAAATTGAATTTAGATAACGTTGAGTTAACAAAATATTTGTTTTTTACTGGTAAAGGT |
| | GGCGTAGGCAAAACAACGATATCAAGTTTTATTGCTTTAAACTTAGCAGAGAATGGAAAGAAAGTAGCTTTAGTAAGTACTGATCC |
| | AGCTAGTAATTTACAAGATGTATTTCAAATGGAATTATCTAATAAATTAACTAAATATCAACCTATACCTAATCTCTCTATAGCCAA |
| | TTTTGACCCGATTGCTGCTGCAGACGATTATAAAGCACAATCTATAGAACCTTATGAGGGTATTCTACCAGAAGATGTGCTTGCTGA |
| | GATGAAAGAACAGTTAAGTGGTTCATGTACAGTTGAAGTAGCAGCATTTAATGAATTTACAAATTTTTTATCCGATAAAACTTTAGA |
| | ACAAGAATTTGATTTCATTATATTTGATACAGCTCCAACAGGTCACACCTTGAGAATGCTTGAATTACCTTCTGCATGGACAGATTA |
| | TTTAAATACAACGAGTAATGACGCTTCTTGCTTAGGTCAATTATCAGGTTTAAATGAAAATAGAGTTAAATATAATTCAGCACTTGA |
| | AAAACTACGTAACCAAGATGATACGACCATGATGTTAGTTGCGAGACCTAGTCACTCTTCTATATATGAAATTCAAAGAGCGCAAC |
| | AAGAATTACAACAACTGTCAATTTCTAAATTCAAAGTAATCATTAACAACTATATAGAAGAAAGTCACGGTTTAATTTCGAGTCAG |
| | ATGAAATCAGAACAAGATAAAAACATTAATCATTTTACTGAATGGTTAAATAACAATCATGCTTATTACGTTCCATATAAAAAGCA |
| | GAAAGAAGAAGGTATAGAAAGTTTAACTAATCTATTAAATGATGATAACTTAATTGAAAATGATGACTTTATTGTTGAAGATCATC |
| | CGCAATTCAATAAATTAATAGATGAAATTGAAAATAGTAAAGTTCAATATTTATTTACAATGGGAAAAGGTGGCGTTGGTAAAACG |
| | ACAGTAGCAACGCAATTAGCTACAGTATTATCTAATAAAGGATATCGTGTTCTTTTAGCAACTACTGACCCTACTAAAGAAATTAAT |
| | GTTGAAACCACAAGTAATTTAAATACTGCTTATATTGATGAAGAACAAGCATTAGAAAAATATAAAAAAGAAGTACTAGCAACAGT |
| | GAATGATGATACACCACAAGACGATATTGATTATATTATGGAAGATTTAAAATCACCTTGTACAGAAGAAATAGCATTTTTCAAAG |
| | CCTTTAGTGACATTATGGAGAATCAAGAAGACATGGATTACGTAATTGTAGATACAGCTCCTACAGGCCATACCTTGCTATTACTTG |
| | ATTCTAGTGAAAATCATCATAGAGAATTAAAGAAAAAATCCACTCAAACTACCAGTAATGTTGAAACATTATTACCCAAAATTCAA |
| | AATAAAAATTTAACACAGATGATAATCGTAACATTAGCAGAAAAAACACCTTATTTAGAATCTAAACGTTTAGTAGAAGATTTAAA |
| | TAGAGCTAATATAGGCCATAATTGGTGGGTTGTTAATCAATCGTTAGTTACGCTAAATCAACGTGATGACCTTTTTAGTAACAAAAA |
| | AGAAGATGAATCATTTTGGATAAACAAGATTAAAAATGAAAGTCTTGATAATTACTTTGTCATACCTTATCGAGTATTAGAATATTG |
| | A |
| SEQ ID NO: 77 | ATGGAGGATGCTGTGGTGGAGATGGATGCTGTTAAGTACTTAAATAAATTGAATTTAGATAACGTTGAGTTAACAAAATATTTGTTT |
| | TTTACTGGTAAAGGTGGCGTAGGCAAAACAACGATATCAAGTTTTATTGCTTTAAACTTAGCAGAGAATGGAAAGAAAGTAGCTTT |
| | AGTAAGTACTGATCCAGCTAGTAATTTACAAGATGTATTTCAAATGGAATTATCTAATAAATTAACTAAATATCAACCTATACCTAA |
| | TCTCTCTATAGCCAATTTTGACCCGATTGCTGCTGCAGACGATTATAAAGCACAATCTATAGAACCTTATGAGGGTATTCTACCAGA |
| | AGATGTGCTTGCTGAGATGAAAGAACAGTTAAGTGGTTCATGTACAGTTGAAGTAGCAGCATTTAATGAATTTACAAATTTTTTATC |
| | CGATAAAACTTTAGAACAAGAATTTGATTTCATTATATTTGATACAGCTCCAACAGGTCACACCTTGAGAATGCTTGAATTACCTTC |
| | TGCATGGACAGATTATTTAAATACAACGAGTAATGACGCTTCTTGCTTAGGTCAATTATCAGGTTTAAATGAAAATAGAGTTAAATA |
| | TAATTCAGCACTTGAAAAACTACGTAACCAAGATGATACGACCATGATGTTAGTTGCGAGACCTAGTCACTCTTCTATATATGAAAT |
| | TCAAAGAGCGCAACAAGAATTACAACAACTGTCAATTTCTAAATTCAAAGTAATCATTAACAACTATATAGAAGAAAGTCACGGTT |
| | TAATTTCGAGTCAGATGAAATCAGAACAAGATAAAAACATTAATCATTTTACTGAATGGTTAAATAACAATCATGCTTATTACGTTC |
| | CATATAAAAAGCAGAAAGAAGAAGGTATAGAAAGTTTAACTAATCTATTAAATGATGATAACTTAATTGAAAATGATGACTTTATT |
| | GTTGAAGATCATCCGCAATTCAATAAATTAATAGATGAAATTGAAAATAGTAAAGTTCAATATTTATTTACAATGGGAAAAGGTGG |
| | CGTTGGTAAAACGACAGTAGCAACGCAATTAGCTACAGTATTATCTAATAAAGGATATCGTGTTCTTTTAGCAACTACTGACCCTAC |
| | TAAAGAAATTAATGTTGAAACCACAAGTAATTTAAATACTGCTTATATTGATGAAGAACAAGCATTAGAAAAATATAAAAAAGAA |
| | GTACTAGCAACAGTGAATGATGATACACCACAAGACGATATTGATTATATTATGGAAGATTTAAAATCACCTTGTACAGAAGAAAT |
| | AGCATTTTTCAAAGCCTTTAGTGACATTATGGAGAATCAAGAAGACATGGATTACGTAATTGTAGATACAGCTCCTACAGGCCATA |
| | CCTTGCTATTACTTGATTCTAGTGAAAATCATCATAGAGAATTAAAGAAAAAATCCACTCAAACTACCAGTAATGTTGAAACATTAT |
| | TACCCAAAATTCAAAATAAAAATTTAACACAGATGATAATCGTAACATTAGCAGAAAAAACACCTTATTTAGAATCTAAACGTTTA |
| | GTAGAAGATTTAAATAGAGCTAATATAGGCCATAATTGGTGGGTTGTTAATCAATCGTTAGTTACGCTAAATCAACGTGATGACCTT |
| | TTTAGTAACAAAAAAGAAGATGAATCATTTTGGATAAACAAGATTAAAAATGAAAGTCTTGATAATTACTTTGTCATACCTTATCGA |
| | GTATTAGAATATTGA |
| SEQ ID NO: 78 | ATGGATGCTGTTAAGTACTTAAATAAATTGAATCCAGATAACATTGAGTTAACAAAATATTTGTTTTTTACTGGTAAAGGTGGCGTA |
| | GGCAAAACAACGATATCAAGTTTTATTGCTTTAAACTTAGCAGAGAATGGAAAGAAAGTAGCTTTAGTAAGTACTGATCCAGCTAG |
| | TAATTTACAAGATGTATTTCAAATGGAATTATCTAATAAATTAACTAAATATCAACCTATACCTAATCTCTCTATAGCCAATTTTGAC |
| | CCGATTGCTGCTGCAGACGATTATAAAGCACAATCTATAGAACCTTATGAGGGTATTCTACCAGAAGATGTGCTTGCTGAGATGAA |
| | AGAACAGTTAAGTGGTTCATGTACAGTTGAAGTAGCAGCATTTAATGAATTTACAAATTTTTTATCCGATAAAACTTTAGAACAAGA |
| | ATTTGATTTCATTATATTTGATACAGCTCCAACAGGTCACACCTTGAGAATGCTTGAATTACCTTCTGCATGGACAGATTATTTAAAT |
| | ACAACGAGTAATGACGCTTCTTGCTTAGGTCAATTATCAGGTTTAAATGAAAATAGAGTTAAATATAATTCAGCACTTGAAAAACT |
| | ACGTAACCAAGATGATACGACCATGATGTTAGTTGCGAGACCTAGTCACTCTTCTATATATGAAATTCAAAGAGCGCAACAAGAAT |
| | TACAACAACTGTCAATTTCTAAATTCAAAGTAATCATTAACAACTATATAGAAGAAAGTCACGGTTTAATTTCGAGTCAGATGAAAT |
| | CAGAACAAGATAAAAACATTAATCATTTTACTGAATGGTTAAATAACAATCATGCTTATTACGTTCCATATAAAAAGCAGAAAGAA |
| | GAAGGTATAGAAAGTTTAACTAATCTATTAAATGATGATAACTTAATTGAAAATGATGACTTTATTGTTGAAGATCATCCGCAATTC |
| | AATAAATTAATAGATGAAATTGAAAATAGTAAAGTTCAATATTTATTTACAATGGGAAAAGGTGGCGTTGGTAAAACGACAGTAGC |
| | AACGCAATTAGCTACAGTATTATCTAATAAAGGATATCGTGTTCTTTTAGCAACTACTGACCCTACTAAAGAAATTAATGTTGAAAC |
| | CACAAGTAATTTAAATACTGCTTATATTGATGAAGAACAAGCATTAGAAAAATATAAAAAAGAAGTACTAGCAACAGTGAATGAT |
| | GATACACCACAAGACGATATTGATTATATTATGGAAGATTTAAAATCACCTTGTACAGAAGAAATAGCATTTTTCAAAGCCTTTAGT |
| | GACATTATGGAGAATCAAGAAGACATGGATTACGTAATTGTAGATACAGCTCCTACAGGCCATACCTTGCTGTTACTTGATTCTAGT |
| | GAAAATCATCATAGAGAATTAAAGAAAAAATCAACTCAAACTACCAGTAATGTTGAAACATTATTACCCAAAATTCAAAATAAAA |
| | ATTTAACACAGATGATAATTGTAACATTAGCAGAAAAAACACCTTATTTAGAATCTAAACGTTTAGTAGAAGATTTAAATAGAGCT |
| | AATATAGGCCATAATTGGTGGGTTGTTAATCAATCGTTAGTTACGCTAAATCAACGTGATGACCTTTTTAGTAACAAAAAAGAAGAT |
| | GAATCATTTTGGATAAACAAGATTAAAAATGAAAGTCTTGATAATTACTTTGTCATACCTTATCGAGTATTAGAATATTGA |
| SEQ ID NO: 79 | ATGGATGCTGTTAAGTACTTAAATAAATTGAATTTAGATAACGTTGAGTTAACAAAATATTTGTTTTTTACTGGTAAAGGTGGCGTA |
| | GGCAAAACAACGATATCAAGTTTTATTGCTTTAAACTTAGCAGAGAATGGAAAGAAAGTAGCTTTAGTAAGTACTGATCCAGCTAG |
| | TAATTTACAAGATGTATTTCAAATGGAATTATCTAATAAATTAACTAAATATCAACCTATACCTAATCTCTCTATAGCCAATTTTGAC |
| | CCGATTGCTGCTGCAGACGATTATAAAGCACAATCTATAGAACCTTATGAGGGTATTCTACCAGAAGATGTGCTTGCTGAGATGAA |
| | AGAACAGTTAAGTGGTTCATGTACAGTTGAAGTAGCAGCATTTAATGAATTTACAAATTTTTTATCCGATAAAACTTTAGAACAAGA |
| | ATTTGATTTCATTATATTTGATACAGCTCCAACAGGTCACACCTTGAGAATGCTTGAATTACCTTCTGCATGGACAGATTATTTAAAT |
| | ACAACGAGTAATGACGCTTCTTGCTTAGGTCAATTATCAGGTTTAAATGAAAATAGAGTTAAATATAATTCAGCACTTGAAAAACT |
| | ACGTAACCAAGATGATACGACCATGATGTTAGTTGCGAGACCTAGTCACTCTTCTATATATGAAATTCAAAGAGCACAACAAGAAT |
| | TACAACAACTGTCAATTTCTAAATTCAAAGTAATCATTAACAACTATATAGAAGAAAGTCACGGTTTAATTTCGAGTCAGATGAAAT |
| | CAGAACAAGATAAAAACATTAATCATTTTACTGAATGGTTAAATAACAATCATGCTTATTACGTTCCATATAAAAAGCAGAAAGAA |
| | GAAGGTATAGAAAGTTTAACTAATCTATTAAATGATGATAACTTAATTGAAAATGATGACTTTATTGTTGAAGATCATCCGCAATTC |
| | AATAAATTAATAGATGAAATTGAAAATAGTAAAGTTCAATATTTATTTACAATGGGAAAAGGTGGCGTTGGTAAAACGACAGTAGC |
| | AACGCAATTAGCTACAGCATTATCTAATAAAGGATATCGTGTTCTTTTAGCAACTACTGACCCTACTAAAGAAATTAATGTTGAAAC |
| | CACAAGTAATTTAAATACTGCTTATATTGATGAAGAACAAGCATTAGAAAAATATAAAAAAGAAGTACTAGCAACAGTGAATGAT |
| | GATACACCACAAGACGATATTGATTATATTATGGAAGATTTAAAATCACCTTGTACAGAAGAAATAGCATTTTTCAAAGCCTTTAGT |
| | GACATTATGGAGAATCAAGACGACATGGATTACGTAATTGTAGATACAGCTCCTACAGGCCATACCTTGCTGTTACTTGATTCTAGT |
| | GAAAATCATCATAGAGAATTAAAGAAAAAATCAACTCAAACTACCAGTAATGTTGAAACATTATTACCCAAAATTCAAAATAAAA |
| | ATTTAACACAGATGATAATTGTAACATTAGCAGAAAAAACACCTTATTTAGAATCTAAACGTTTAGTAGAAGATTTAAATAGAGCT |
| | AATATAGGCCATAATTGGTGGGTTGTTAATCAATCGTTAGTTACGCTAAATCAACGTGATGACCTTTTTAGTAACAAAAAAGAAGAT |
| | GAATCATTTTGGATAAACAAGATTAAAAATGAAAGTCTTGATAATTACTTTGTCATACCTTATCGAGTATTAGAATATTGA |
| SEQ ID NO: 80 | TTGAATTTAGATAACGTTGAGTTAACAAAATATTTGTTTTTTACTGGTAAAGGTGGCGTAGGCAAAACAACGATATCAAGTTTTATT |
| | GCTTTAAACTTAGCAGAGAATGGAAAGAAAGTAGCTTTAGTAAGTACTGATCCAGCTAGTAATTTACAAGATGTATTTCAAATGGA |
| | ATTATCTAATAAATTAACTAAATATCAACCTATACCTAATCTCTCTATAGCCAATTTTGACCCGATTGCTGCTGCAGACGATTATAA |
| | AGCACAATCTATAGAACCTTATGAAGGTATTCTACCAGAAGATGTGCTTGCTGAGATGAAAGAACAGTTAAGTGGTTCATGTACAG |
| | TTGAAGTAGCAGCATTTAATGAATTTACAAATTTTTTATCCGATAAAACTTTAGAACAAGAATTTGATTTCATTATATTTGATACAGC |
| | TCCAACAGGTCATACCTTGAGAATGCTTGAATTACCTTCTGCATGGACAGATTATTTAAATACAACGAGTAATGACGCTTCTTGCTT |
| | AGGTCAATTATCAGGTTTAAATGAAAATAGAGATAAATATAATTCAGCACTTGAAAAACTACGTAACCAAGATGATACGACCATGA |
| | TGTTAGTTGCGAGACCTAGTCACTCTTCTATATATGAAATTCAAAGAGCGCAACAAGAATTACAACAACTGTCAATTTCTAAATTCA |
| | AAGTAATCATTAACAACTATATAGAAGAAAGTCACGGTTTAATTTCGAGTCAGATGAAATCAGAACAAGATAAAAACATTAATCAT |
| | TTTACTGAATGGTTAAATAACAATCATGCTTATTACGTTCCATATAAAAAGCAGAAAGAAGAAGGTATAGAAAGTTTAACTAATCT |
| | ATTAAATGATGATAACTTAATTGAAAATGATGACTTTATTGTTGAAGATCATCCGCAATTCAATAAATTAATAGATGAAATTGAAAA |
| | TAGTAAAGTTCAATATTTATTTACAATGGGAAAAGGTGGCGTTGGTAAAACGACAGTAGCAACGCAATTAGCTACAGCATTATCTA |
| | ATAAAGGATATCGTGTTCTTTTAGCAACTACTGACCCTACTAAAGAAATTAACGTTGAAACTACAAGTAATTTAAATACTGCTTATA |
| | TTGATGAAGAACAAGCATTAGAAAAGTATAAAAAAGAAGTACTAGCCACAGTGAATGATGATACACCACAAGACGATATTGATTA |
| | TATTATGGAAGATTTAAAATCACCTTGTACAGAAGAAATAGCATTTTTCAAAGCCTTTAGTGACATTATGGAGAATCAAGACGACA |
| | TGGATTACGTAATTGTAGATACAGCTCCTACAGGCCATACCTTGCTGTTACTTGATTCTAGTGAAAATCATCATAGAGAATTAAAGA |
| | AAAAATCAACTCAAACTACCAGTAATGTTGAAACATTATTACCCAAAATTCAAAATAAAAATTTAACACAGATGATAATTGTAACA |
| | TTAGCAGAAAAAACACCTTATTTAGAATCTAAACGTTTAGTAGAAGATTTAAATAGAGCTAATATAGGCCATAATTGGTGGGTTGTT |
| | AATCAATCGTTAGTTACGCTAAATCAACGTGATGACCTTTTTAGTAACAAAAAAGAAGATGAATCATTTTGGATAAACAAGATTAA |
| | AAATGAAAGTCTTGATAATTACTTTGTCATACCTTATCGAGTATTAGAATATTGA |
| SEQ ID NO: 81 | GTGGAAATGGATGCTGTTAAATACTTAAATAAATTGAATTTAGATAACATTGAGTTAACAAAATATTTGTTTTTTACTGGTAAAGGT |
| | GGCGTAGGCAAAACAACGATATCAAGTTTTATTGCTTTAAACTTAGCAGAGAATGGAAAGAAAGTAGCTTTAGTAAGTACTGATCC |
| | AGCTAGTAATTTACAAGATGTATTTCAAATGGAATTATCTAATAAATTAACTAAATATCAACCTATACCTAATCTCTCTATAGCCAA |
| | TTTCGACCCGATTGTTGCTGCAGACGATTATAAAGCACAATCTATAGAACCTTATGAGGGTATTCTACCAGAAGATGTGCTTGCTGA |
| | AATGAAAGAACAGTTAAGTGGTTCATGTACAGTTGAAGTAGCAGCATTTAATGAATTTACAAATTTTTTATCCGATAAAACTTTAGA |
| | ACAAGAATTTGATTTCATTATATTTGATACAGCTCCCACAGGTCACACTTTGAGAATGCTTGAATTACCTTCTGCATGGACAGATTA |
| | TTTAAATACAACGAGTAATGACGCTTCTTGCTTAGGTCAATTATCAGGTTTAAATGAAAATAGAGTTAAATATAATTCAGCACTTGA |
| | AAAACTACGTAACCAAGATGATACGACCATGATGTTAGTTGCGAGACCTACTCACTCTTCTATATATGAAATTCAAAGAGCGCAAC |
| | AAGAATTACAACAACTGTCAATTTCTAAATTCAAAGTAATCATTAACAACTATATAGAAGAAAGTCACGGTTTAATTTCGAGTCAG |
| | ATGAAATCGGAACAAGATAAAAACATTAATCATTTTACTGAATGGTTAAATAACAATCATGCTTATTACGTTCCATATAAAAATCA |
| | GAAAGAAGAAGGTATAGAAAATTTAACTAATCTATTAAATGATGATAACTTAATTGAAAATGATGACTTTATTGTTGAAGATCATC |
| | CGCAATTCAATAAATTAATCGATGAAATTGAAAATAGTAAAGTTCAATATTTATTTACAATGGGAAAAGGTGGCGTTGGTAAAACG |
| | ACAGTAGCAACGCAATTAGCTACAACGTTATCTAATAAAGGATATCGTGTTCTTTTAGCAACTACTGACCCTACTAAAGAAATTAAT |
| | GTTGAAACTACAAGTAATTTAAATACTGCTTATATTGATGAAGAACAAGCATTAGAAAAGTATAAAAAAGAAGTACTAGCCACAGT |
| | GAATGATGATACACCACAAGACGATATTGATTATATTATGGAAGATTTAAAATCACCTTGTACAGAAGAAATAGCATTTTTCAAAG |
| | CCTTTAGTGACATTATGGAGAATCAAGACGACATGGATTACGTCATTGTAGATACAGCTCCTACAGGCCATACCTTGCTGTTACTTG |
| | ATTCTAGTGAAAATCATCATAGAGAATTAAAGAAAAAATCAACTCAAACTACCAGTAATGTTGAAACATTATTACCTAAAATTCAA |
| | AATAAAAATTTAACACAGATGATAATCGTAACACTAGCAGAAAAAACACCTTATTTAGAATCTAAACGTTTAGTAGAAGATTTAAA |
| | TAGAGCTAATATAGGCCATAATTGGTGGGTTGTTAATCAATCGTTAGTTACGCTAAATCAACGTGATGACCTTTTTAGTAACAAAAA |
| | AGAAGATGAATCATTTTGGATAAACAAGATTAAAAATGAAAGTTTTGACAATTATTTTGTCATACCTTATGGGGGGTTATCATAA |
| SEQ ID: 97 | GATAGTGTTAAGAAAATTCCTTTTGAAAATATAAAATCAGCCGAGTATGATTCTGACAGAACAATCAATAATAAAGGAAAAGTAAT |
| | CGAATATCATCGAGTCATCGTATCTTATGGAAATGGTGAAGATATAGAATTTAGTTCCGAACAATTTGATAGT |
| SEQ ID: 98 | AAGCATTTAACGATTTAGAGGCACATATAGCCCAAATAAGAGCCAAAAAAGAAGAAGAGGAGGAATATTACCAAAGTTTAAAGAA |
| | TGTTCTGTATGAGCCTCTAAACAACGAATTTGAGTATGAATATAAAAAGCGAAGTTTATTTTCAAAAGAAAGTGAGCCAACAGGAC |
| | GAGTCATTTTGAAAGAAGAAGATTATAAAACATTAAAAGAACAGG |
| SEQ ID: 99 | AATTTCTTATTTGAAAACAATTTTATCTTCATCATAAGCATCAACTGAATAACCTACATATGCATGTGTTTTTAATATTAAATGTTTT |
| | GGTTCGCTTTTAAATTCATATTCATAAATATTAAATTTGAATTCTTCTGTTATTGTTTGGTGTTCTTTTAATTCAACATTTGTGTGTAA |
| | TGTAAATGTTTGTAATTCATTTTCTAAAACAGGAAATTGACT |
| SEQ ID: 100 | AATTTCTTATTTGAAAACAATTTTATCTTCATCATAAGCATCAACTGAATAACCTACATATGCATGTGTTTTTAATATTAAATGTTTT |
| | GGTTCACTTTTAAATTCATATTCATAAATATTAAATTTGAATTCTTCTGTTATTGTGTGGTGTTCTTTTAATTCAACATTTGTGTGTAG |
| | TGTAAATGTTTGTAATTCATTTTCTAAAACAGGAAATTGACT |

A PCR primer set for amplifying a coa gene comprises, for example, the primer sequences SEQ ID NOS: 1 and 3.

A probe for binding to an amplicon(s) of a coa gene, or to a coa gene target, comprises, for example, SEQ ID NO: 2.

A PCR primer set for amplifying a nuc gene comprises, for example, the primer sequences SEQ ID NOS: 106 and 108.

A probe for binding to an amplicon(s) of a nuc gene, or to a nuc gene target, comprises at least one of the following probe sequences: SEQ ID NOS: 107, 109, 110 and 111.

A PCR primer set for amplifying a mecA gene comprises at least one of the following sets of primer sequences (1) SEQ ID NOS: 4 and 6; (2) SEQ ID NOS: 101 and 103; (3) SEQ ID NOS: 101 and 104 and (4) SEQ ID NOS: 101 and 105.

A probe for binding to an amplicon(s) of a mecA gene, or to a mecA gene target, comprises at least one of the following probe sequences: SEQ ID NOS. 5 and 102.

A PCR primer set for amplifying an org region comprises at least one of the following is sets of primer sequences: (1) SEQ ID NOS: 7 and 9; (2) SEQ ID NOS: 11 and 9; (3) SEQ ID NOS: 7 and 13; (4) SEQ ID NOS: 7 and 15; (5) SEQ ID NOS: 11 and 13; and (6) SEQ ID NOS 11 and 15. A lack of an org region amplicon, in the presence of a nuc or coa amplicon, is indicative of a sample that contains MRSA, due to a disruption in the orfX region. The presence of an org region amplicon indicates that the sample does not contain MRSA and most likely, the sample contains MSSA.

The preceding numbering of the six sets of primers does not correspond exactly to the “Group” numbering scheme in Table 4 because certain groups use the same primer set, but different internal probes. For example, Groups 3 and 4 of Table 4 each employ the forward primer of SEQ ID NO:7 and the reverse primer of SEQ ID NO: 9, but different internal probes in each instance, e.g., SEQ ID NOS: 8 and 10. Accordingly, primer set “(1)” of the preceding passage implies any one of Groups 3 or 4 of Table 4.

A probe for binding to an amplicon(s) of an orfX region, or to an orfX region target, comprises at least one of the following probe sequences: SEQ ID NOS: 8, 10, 12, 14 and 16 (orfX probes).

A PCR primer set for amplifying a CoNS specific marker comprises at least one of the following sets of primer sequences: (1) SEQ ID NOS: 17 and 19; (2) SEQ ID NOS: 20 and 22; (3) SEQ ID NOS: 23 and 19; (4) SEQ ID NOS: 24 and 22; (5) SEQ ID NOS: 25 and 27; and (6) SEQ ID NOS: 20 and 28; (7) SEQ ID NOS: 82 and 84; (8) SEQ ID NOS: 85 and 87; (9) SEQ ID NOS: 88 and 90; (10) SEQ ID NOS: 91 and 93 and (11) SEQ ID NOS: 94 and 96.

A probe for binding to an amplicon(s) of a CoNS specific marker, or to a CoNS specific marker target, comprises at least one of the following probe sequences: SEQ ID NOS: 18, 21, 26, 83, 86, 89, 92 and 95 (CoNS specific marker probes).

Any set of primers can be used simultaneously in a multiplex reaction with one or more other primer sets, so that multiple amplicons are amplified simultaneously.

An internal/process control plasmid is added directly to the reaction mix to monitor the integrity of the PCR reagents and the presence of PCR inhibitors. The primers and probes for the process control (Geobacillus stearothermophilus) are disclosed in Table 6.

CoNS specific marker sequences are described in Table 7.

### Example 5

### Testing orfX Primer and Probe Sequences for their Ability to Amplify their Intended Target Sequences

Certain oligonucleotide sequences listed in Table 4 were tested for their ability to amplify their intended target sequences. As a representative example, an oligonucleotide solution comprising SEQ ID NOS: 7, 9 and 10 was used to amplify the orfX region of S. aureus (ATCC No. 35556). This same oligonucleotide solution was included in a polymerase chain reaction (PCR) where genomic DNA (gDNA) isolated from MRSA (ATCC No. 700699) was input as the template and in an additional PCR where water was included as the template (negative control).

Duplicate reactions where S. aureus gDNA was input as the template yielded Ct values indicative of the formation of PCR product, while no Ct values (UND) were obtained in the MRSA and negative control reactions, thus indicating that these primers and probe amplify and specifically detect the orfX region from S. aureus and do not amplify and do not detect the disrupted orfX region in MRSA.

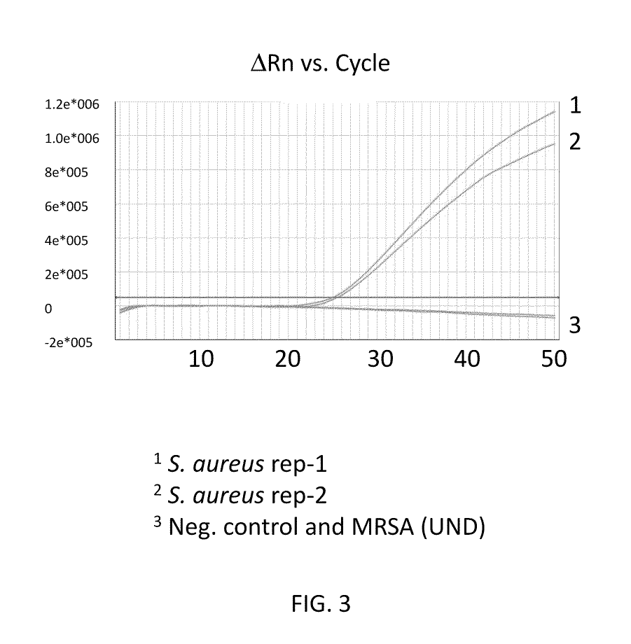

An amplification plot corresponding Ct values listed in Table 8 is shown in FIG. 3.

TABLE 8
Ct values showing presence of the orfX region.

| Input | Ct |
|---|---|
| S. aureus rep-1 | 25.05 |
| S. aureus rep-2 | 25.49 |
| MRSA | UND |
| Neg Ctrl. | UND |

### Example 6

### Testing orfX primer and Probe Sequences for their Specificity in Amplification of their Intended Target Sequences

Certain oligonucleotide sequences listed in Table 4 were tested for their ability to amplify specifically their intended target sequences. As a representative example, an oligonucleotide solution comprising SEQ ID NOS: 7, 9 and 10 was used to amplify the orfX region of S. aureus (ATCC No. 35556). This same oligonucleotide solution was included in PCRs where gDNA isolated from S. aureus (ATCC No. 35556), S. epidermidis (ATCC No. 12228), S. epidermidis (ATCC No. 35984), S. haemolyticus (ATCC No. 29970), and S. hominis (ATCC No. 700236) were input as templates; an additional PCR was run where water was included as the template (negative control). Only the reaction where S. aureus gDNA was input as the template yielded a Ct value indicative of the formation of PCR product; no Ct values (UND) were obtained from the reactions where gDNA from the other Staphylococcus species was input as template or from the negative control reaction, thus indicating that these primers and probe amplify and specifically detect orJX of S. aureus and do not amplify and do not detect orJX of the other tested Staphylococcal species.

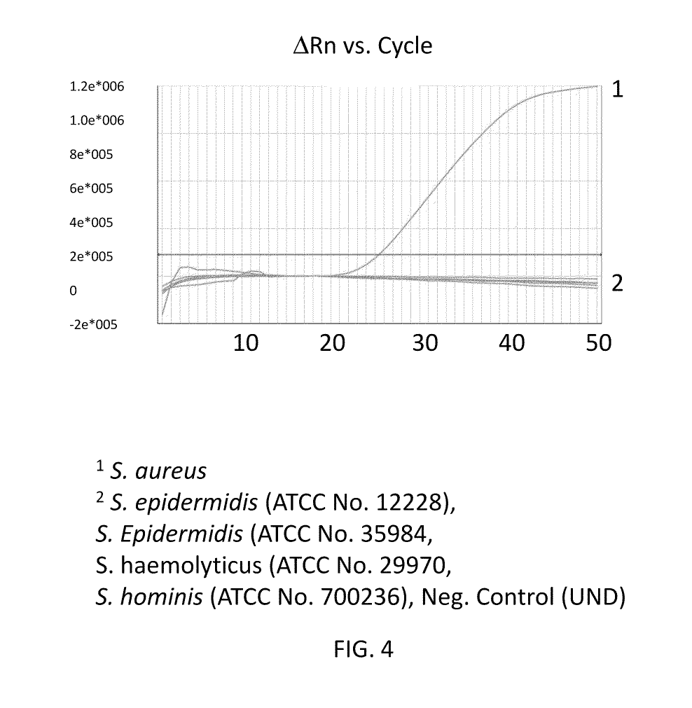

An amplification plot corresponding Ct values listed in Table 9 is shown in FIG. 4.

TABLE 9
Ct values showing specificity for the S. aureus orfX region.

| Input | Ct |
|---|---|
| S. aureus | 22.36 |
| S. epidermidis (ATCC No. 12228) | UND |
| S. epidermidis (ATCC No. 35984) | UND |
| S. haemolyticus (ATCC No. 29970) | UND |
| S. hominis (ATCC No 700236) | UND |
| Neg Ctrl. | UND |

### Example 7

### Testing mecA Primer and Probe Sequences for Amplification of their Intended Target Sequences.

Certain oligonucleotide sequences listed in Table 4 were tested for their ability to amplify their intended target sequences. As a representative example, an oligonucleotide solution comprising SEQ ID NOS: 4, 5 and 6 was used to amplify the mecA gene found in MRSA (ATCC No. 700699 used in this experiment). This oligonucleotide solution was used in additional PCRs where gDNA isolated from S. aureus (MSSA; ATCC No. 35556) or water (negative control) were included as the templates.

Only the reaction where MRSA gDNA was input as the template yielded a Ct value indicative of the formation of PCR product; no Ct values (UND) were obtained from the reactions where MSSA gDNA was input as template or from the negative control reaction, thus indicating that these primers and probe amplify and specifically detect the mecA gene of MRSA.

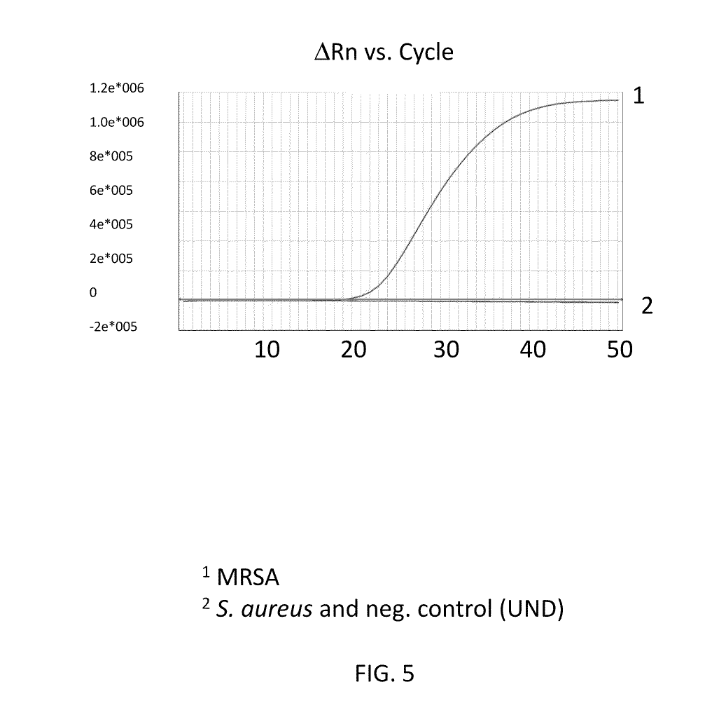

An amplification plot corresponding Ct values are listed in Table 10 is shown in FIG. 5.

TABLE 10
Ct values showing presence of mecA.

| Input | Ct |
|---|---|
| S. aureus | UND |
| MRSA | 19.38 |
| Neg Ctrl. | UND |

### Example 8

### Testing mecA Primer and Probe Sequences for Amplification of their Intended Target Sequences.

Certain oligonucleotide sequences listed in Table 4 were tested for their ability to amplify their intended target sequences. An oligonucleotide solution comprising SEQ ID NOS: 101, 102 and 103 was used to amplify the mecA gene found in MRSA (ATCC No. 700699 used in this experiment). This oligonucleotide solution was used in additional PCRs where gDNA isolated from S. aureus (MSSA; ATCC No. 35556) or water (negative control) were included as the templates. Only the reaction where MRSA gDNA was input as the template yielded a Ct value indicative of the formation of PCR product; no Ct values (UND) were obtained from the reactions where MSSA gDNA was input as template or from the negative control reaction, thus indicating that these primers and probe amplify and specifically detect the mecA gene of MRSA.

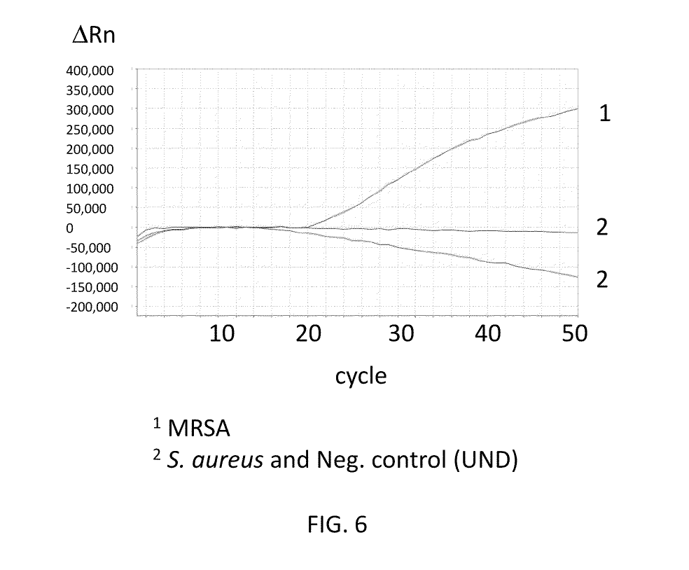

An amplification plot corresponding Ct values listed in Table 11 is shown in FIG. 6.

TABLE 11
Ct values showing presence of mecA.

| Input | Ct |
|---|---|
| S. aureus | UND |
| MRSA | 24.99 |
| Neg Ctrl. | UND |

### Example 9

### Testing coa Primer and Probe Sequences for their Ability to Amplify their Intended Target Sequences

The coa oligonucleotide sequences listed in Table 4 were tested for their ability to amplify their intended target sequences. An oligonucleotide solution comprising SEQ ID NOS: 1, 2 and 3 was used to amplify the coa gene of S. aureus (ATCC No. 35556). This same oligonucleotide solution was included in a PCR where water was included as the template (negative control). Only the reaction where S. aureus gDNA was input as the template yielded a Ct value indicative of the formation of PCR product; no Ct value (UND) was obtained from the negative control reaction thus indicating that these primers and probe amplify and specifically detect the coa gene of S. aureus.

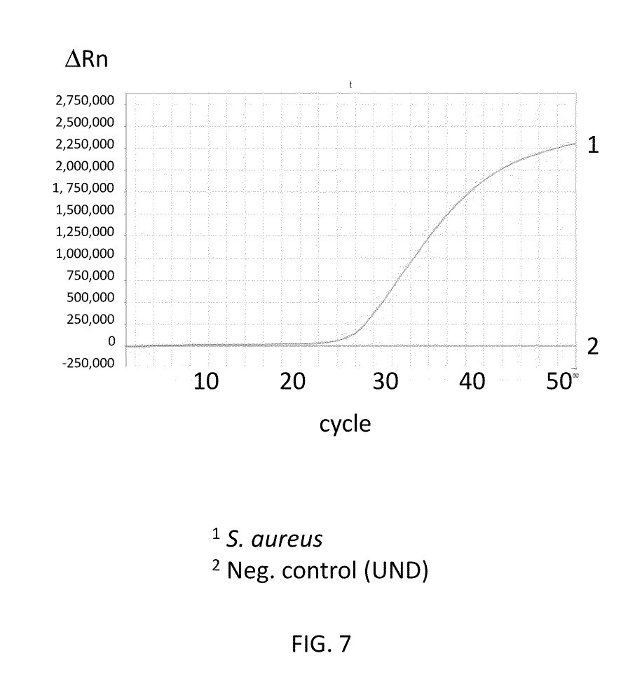

An amplification plot corresponding Ct values listed in Table 12 is shown in FIG. 7.

TABLE 12
Ct values showing presence of coa.

| Input | Ct |
|---|---|
| S. aureus | 22.72 |
| Neg Ctrl. | UND |

### Example 10

### Testing coa Primer and Probe Sequences for their Specificity in Amplification of their Intended Target Sequences

The coa oligonucleotide sequences listed in Table 4 were tested for their ability to amplify specifically their intended target sequences. An oligonucleotide solution consisting of SEQ ID NOS: 1, 2 and 3 was used to amplify the coa gene of S. aureus (MSSA; ATCC No. 35556) and S. aureus (MRSA; ATCC No. 700699); gDNAs isolated from S. epidermidis (ATCC No. 12228), S. epidermidis (ATCC No. 35984), S. haemolyticus (ATCC No. 29970), and S. hominis (ATCC No. 700236) and water (negative control) were also input as templates in additional PCRs. Only the reactions where the S. aureus gDNAs were input as templates yielded a Ct value indicative of the formation of PCR product; no Ct values (UND) were obtained from the reactions where gDNA from the other Staphylococcus species was input as template or from the negative control reaction , thus indicating that these primers and probe amplify and specifically detect the coa gene of S. aureus and do not amplify and do not detect genes from the other tested Staphylococcal species.

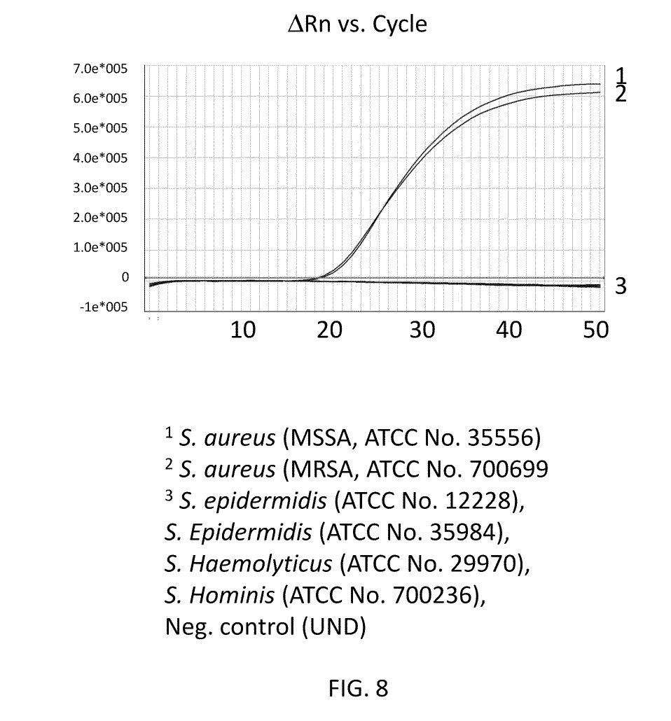

An amplification plot corresponding Ct values listed in Table 13 is shown in FIG. 8.

TABLE 13
Ct values showing specificity for S. aureus coa.

| Input | Ct |
|---|---|
| S. aureus (MSSA, ATCC No. 35556) | 19.65 |
| S. aureus (MRSA; ATCC No. 700699) | 19.26 |
| S. epidermidis (ATCC No. 12228) | UND |
| S. epidermidis (ATCC No. 35984) | UND |
| S. haemolyticus (ATCC No. 29970) | UND |
| S. hominis (ATCC No 700236) | UND |
| Neg Ctrl. | UND |

### Example 11

### Testing nuc Primer and Probe Sequences for their Specificity in Amplification of their Intended Target Sequences

The nuc oligonucleotide sequences listed in Table 4 were tested for their ability to amplify specifically their intended target sequences. An oligonucleotide solution comprising SEQ ID NOS: 106, 107 and 108 was used to amplify nuc gene of S. aureus (MSSA; ATCC No. 35556) and S. aureus (MRSA; ATCC No. 700699); gDNAs isolated from S. epidermidis (ATCC No. 12228), S. epidermidis (ATCC No. 35984), S. haemolyticus (ATCC No. 29970), and S. hominis (ATCC No. 700236) and water (negative control) were also input as templates in additional PCRs. Only the reactions where the S. aureus gDNAs were input as templates yielded Ct values indicative of the formation of PCR product; no Ct values (UND) were obtained from the reactions where gDNA from the other Staphylococcus species was input as template or from the negative control reaction, thus indicating that these primers and probe amplify and specifically detect the nuc gene of S. aureus and do not amplify and do not detect any genes from the other tested Staphylococcal species.

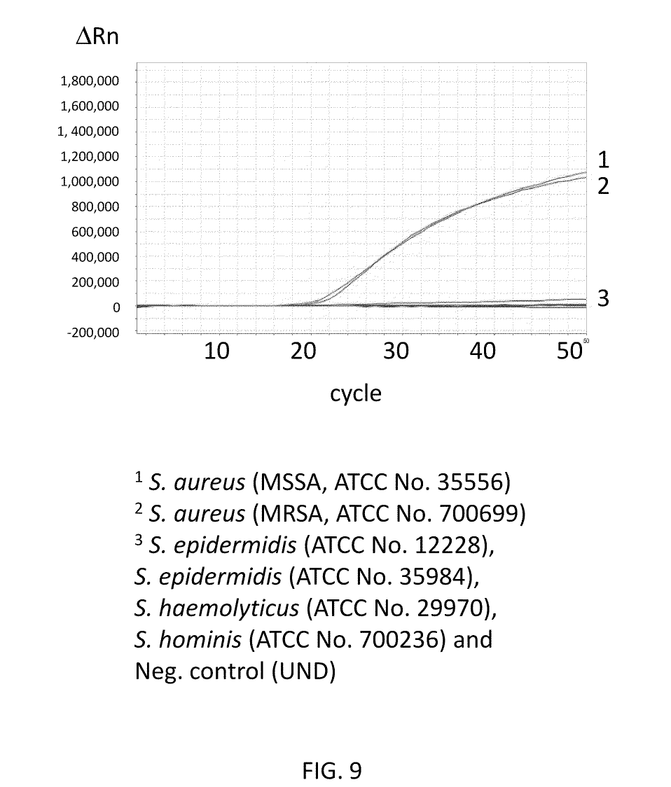

An amplification plot corresponding Ct values listed in Table 14 is shown in FIG. 9.

TABLE 14
Ct values showing specificity for S. aureus nuc.

| Input | Ct |
|---|---|
| S. aureus (MSSA, ATCC No. 35556) | 21.84 |
| S. aureus (MRSA; ATCC No. 700699) | 20.93 |
| S. epidermidis (ATCC No. 12228) | UND |
| S. epidermidis (ATCC No. 35984) | UND |
| S. haemolyticus (ATCC No. 29970) | UND |
| S. hominis (ATCC No 700236) | UND |
| Neg Ctrl. | UND |

### Example 12

### Testing CoNS Marker Primer and Probe Sequences for their Specificity in Amplification of their Intended Target Sequences

Certain oligonucleotide sequences listed in Table 4 were tested for their ability to amplify specifically their intended target sequences. As a representative example, an oligonucleotide solution comprising SEQ ID NOS: 17, 18, 19 was used to amplify a novel CoNS marker in S. epidermidis (ATCC No 12228) and S. epidermidis (ATCC No. 35984). This same oligonucleotide solution was included in a PCR where gDNA isolated from S. aureus (MSSA; ATCC No. 35556) and S. aureus (MRSA; ATCC No. 700699) was input as the template and an additional PCR where water was included as the template (negative control).

Only the reactions where the S. epidermidis gDNAs were input as templates yielded Ct values indicative of the formation of PCR product; no Ct values were obtained from the reactions where gDNA from the other S. aureus strains was input as template or from the negative control reaction, thus indicating that these primers and probe amplify and specifically detect a CoNS specific marker gene and do not amplify and do not detect any gene of S. aureus.

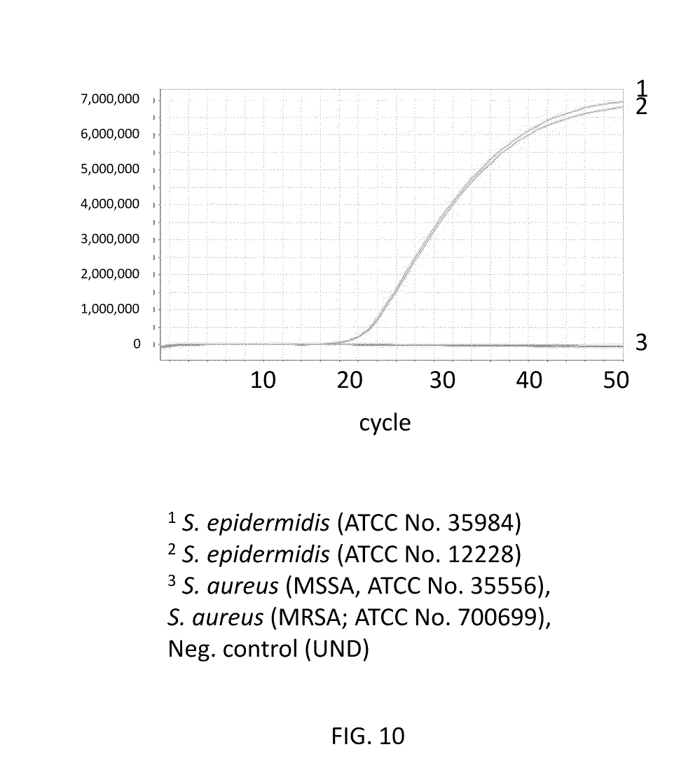

An amplification plot corresponding Ct values listed in Table 15 is shown in FIG. 10.

TABLE 15
Ct values showing specificity for CoNS marker

| Input | Ct |
|---|---|
| S. aureus (MSSA, ATCC No. 35556) | UND |
| S. aureus (MRSA; ATCC No. 700699) | UND |
| S. epidermidis (ATCC No. 12228) | 20.89 |
| S. epidermidis (ATCC No. 35984) | 20.97 |
| Neg Ctrl. | UND |

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

What is claimed is:

1. A method of detecting a methicillin-resistant S. aureus or methicillin-sensitive S. aureus in a biological sample, comprising the steps of:
a) contacting a biological sample with a first oligonucleotide set designed to amplify and/or detect a S. aureus coa or nuc gene; a second oligonucleotide set designed to amplify and/or detect a S. aureus mecA gene; and a third oligonucleotide set designed to amplify and/or detect a S. aureus orfX region, wherein the third oligonucleotide set will not amplify or detect the orfX region if the orfX gene comprises an insertion sequence; and
b) performing a nucleic acid amplification on the contacted sample, wherein amplification and/or detection of a product from both the first and second oligonucleotide sets and no amplification and/or no detection of a product from the third oligonucleotide set indicates the presence of methicillin-resistant S. aureus in the sample, and wherein amplification and/or detection of a product from both the first and third oligonucleotide sets and no amplification and/or no detection of a product from the second oligonucleotide set indicates the presence of methicillin-sensitive S. aureus in the sample.

2. The method of claim 1, wherein the first oligonucleotide set is designed to amplify a S. aureus coa or nuc gene, and comprises one or more oligonucleotides comprising one or more sequences selected from the group consisting of SEQ ID NOS: 1, 3, 106 and 108.

3. The method of claim 1, wherein the third oligonucleotide set is designed to amplify a S. aureus orfX region, and comprises one or more oligonucleotides comprising one or more sequences selected from the group consisting of SEQ ID NOS: 7, 9, 11, 13 and 15.

4. The method of claim 1, wherein the second oligonucleotide set is designed to amplify a S. aureus mecA gene, and comprises one or more oligonucleotides comprising one or more sequences selected from the group consisting of SEQ ID NOS: 4, 6, 101 and 103-105.

5. The method of claim 1, wherein the first oligonucleotide set comprises a probe that detects a S. aureus coa or nuc gene amplicon, wherein the probe comprises a sequence selected from the group consisting of: SEQ ID NOS: 2, 107 and 109-111.

6. The method of claim 1, wherein the second oligonucleotide set comprises a probe that detects a S. aureus mecA gene amplicon, wherein the probe comprises a sequence selected from the group consisting of: SEQ ID NOS: 5 and 102.

7. The method of claim 1, wherein the third oligonucleotide set comprises a probe that detects a S. aureus orfX region amplicon, wherein the probe comprises a sequence selected from the group consisting of: SEQ ID NOS: 8, 10, 12, 14 and 16.

8. A method of hybridizing one or more nucleic acid sequences comprising a sequence selected from the group consisting of: SEQ ID NOS: 1-28, 82-96, 101-111 to one or more target nucleic acids selected from the group consisting of: a methicillin resistance gene, is an org region, a coa gene, a nuc gene, a CoNS specific marker gene, and combinations thereof, the method comprising contacting a sample comprising the target nucleic acid with the one or more nucleic acid sequences under conditions suitable for hybridization.

9. The method of claim 8, further comprising isolating the one or more hybridized target nucleic acids.

10. The method of claim 8, further comprising quantitating the extent of hybridization of the one or more nucleic acids to the one or more target nucleic acids.

11. The method of claim 8, further comprising sequencing the one or more hybridized target nucleic acids.

12. The method of claim 8, further comprising monitoring and/or screening for the presence of the one or more hybridized target nucleic acids.

13. A method of producing a nucleic acid product, comprising contacting one or more nucleic acid sequences selected from the group consisting of: SEQ ID NOS: 1, 3, 4, 6, 7, 9, 11, 13, 15, 17, 19, 20, 22-25, 27-29, 31, 36, 38, 40, 43, 45, 46, 48, 49, 51, 52, 56, 59, 60, 64-67, 69-72, 82, 84, 85, 87, 88, 90, 91, 93, 94, 96, 101, 103-106 and 108, to a template nucleic acid from a target selected from the group consisting of: a methicillin—resistance gene, an orfX region, a coa gene, a nuc gene, a CoNS specific marker gene, a G. Stearothermophilus marker gene, and combinations thereof, under conditions suitable for nucleic acid polymerization.

14. The method of claim 13, wherein the nucleic acid product is an amplicon produced using at least one forward primer selected from the group consisting of: SEQ ID NOS: 1 (coa); 4, 101 (mecA); 106 (nuc); 7 and 11 (orfX region); and 17, 20, 23-25, 82, 85, 88, 91 and 94 (CoNS specific marker); and at least one reverse primer selected from the group consisting of: SEQ ID NOS: 3 (coa); 6, 103, 104 and 105 (mecA); 108 (nuc); 9, 13 and 15 (orJX region); and 19, 22, 27, 28, 84, 87, 90, 93 and 96 (CoNS specific marker).

15. A probe or set of probes that hybridizes to the nucleic acid product of claim 13.

16. The probe of claim 15, wherein the probe or set of probes comprises one or more sequences selected from the group consisting of: SEQ ID NOS: 2 (coa); 5, 102 (mecA); 107, 109, 110, 111 (nuc); 8, 10, 12, 14, 16 (orJX region); 18, 21, 26, 83, 86, 89, 92 and 95 (CoNS specific marker).

17. The probe or set of probes of claim 15, wherein each probe sequence is labeled with a detectable label that is different from a detectable label associated with a different probe sequence.

18. The probe of claim 17, wherein the probe is labeled with a detectable label selected from the group consisting of: a fluorescent label, a chemiluminescent label, a quencher, a radioactive label, biotin and gold.

19. A method for detecting or screening for a methicillin resistance gene or an orJX region or a coa gene or a nuc gene or a CoNS specific marker gene in a sample, comprising:
a) contacting the sample with at least one forward primer comprising a sequence selected from the group consisting of: SEQ ID NOS: 1 (coa); 4, 101 (mecA); 106 (nuc); 7 and 11 (orfX region); and 17, 20, 23, 24, 25, 82, 85, 88, 91 and 94 (CoNS specific marker); and at least one reverse primer selected from the group consisting of SEQ ID NOS: 3 (coa); 6, 103, 104 and 105 (mecA); 108 (nuc); 9, 13 and 15 (orfX region); and 19, 22, 27, 28, 84, 87, 90, 93 and 96 (CoNS specific marker) under conditions such that nucleic acid amplification occurs to yield an amplicon; and
b) contacting the amplicon with one or more probes comprising one or more sequences selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker) under conditions such that hybridization of the probe to the amplicon occurs;
wherein hybridization of the probe is indicative of a methicillin resistance gene or orfX is region or coa gene or nuc gene or CoNS specific marker sequence in the sample.

20. The method of claim 19, wherein the at least one forward primer, the at least one reverse primer and the one or more probes are selected from the group consisting of: Groups 1-16, 74-80 of Table 4 and Groups 17-22, 69-73 of Table 5.

21. A kit for detecting or screening for a methicillin-resistance gene or orfX region or a coa gene or a nuc gene or a CoNS specific marker sequence in a sample, comprising one or more probe sequences comprising a sequence selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker).

22. The kit of claim 21, further comprising:
a) at least one forward primer comprising a sequence selected from the group consisting of: SEQ ID NOS: 1 (coa); 4, 101 (mecA); 106 (nuc); 7 and 11 (orfX);
and 17, 20, 23, 24, 25, 82, 85, 88, 91 and 94 (CoNS specific marker); and
b) at least one reverse primer comprising a sequence selected from the group consisting of: SEQ ID NOS: 3 (coa); 6, 103, 104 and 105 (mecA); 108 (nuc); 9, 13 and 15 (orfX); and 19, 22, 27, 28, 84, 87, 90, 93 and 96 (CoNS specific marker).

23. The kit of claim 21, further comprising reagents for quantitating, screening and/or sequencing a methicillin-resistance gene or orfX region or a coa gene or a nuc gene or a CoNS specific marker sequence in the sample.

24. The kit of claim 21, wherein the one or more probe sequences are labeled with different detectable labels.

21. he kit of claim 21, wherein the one or more probe sequences are labeled with the same detectable label.

26. The kit of claim 21, further comprising an internal control and/or process control.

27. The kit of claim 21, wherein the at least one forward primer and the at least one reverse primer are selected from the group consisting of: Groups 1-16, 74-80 of Table 4, and is Groups 17-22, 69-73 of Table 5.

28. A probe that hybridizes to a methicillin resistance gene target or an orfX region or a coa gene or a nuc gene or a CoNS specific marker sequence target.

29. The probe of claim 28, wherein the probe comprises a sequence selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92, 95 (CoNS specific marker).

30. The probe of claim 28, wherein the probe is labeled with a detectable label selected from the group consisting of: a fluorescent label, a chemiluminescent label, a quencher, a radioactive label, biotin and gold.

31. An isolated or synthesized nucleic acid sequence comprising a sequence selected from the group consisting of: SEQ ID NOS: 1-111.

32. A primer set comprising at least one forward primer selected from the group consisting of: SEQ ID NOS: 1 (coa); 4, 101 (mecA); 106 (nuc); 7 and 11 (orfX region); and 17, 20, 23, 24, 25, 82, 85, 88, 91 and 94 (CoNS specific marker); and at least one reverse primer selected from the group consisting of: SEQ ID NOS: 3 (coa); 6, 103, 104 and 105 (mecA); 108 (nuc); 9, 13 and 15 (orfX region); and 19, 22, 27, 28, 84, 87, 90, 93 and 96 (CoNS specific marker).

33. The primer set of claim 32, wherein the primer set is selected from the group consisting of: Groups 1-16, 74-80 of Table 4, Groups 17-22, 69-73 of Table 5, and Groups 23-68 of Table 6.

34. A method of diagnosing a condition, syndrome, colonization or disease in a human associated with a methicillin-resistant organism or an orfX region or a coa gene or a nuc gene or a CoNS specific marker sequence comprising:
a) contacting a sample with at least one forward and reverse primer set selected from the group consisting of: Groups 1-16, 74-80 of Table 4, and Groups 17-22, 69-73 is of Table 5;
b) conducting an nucleic acid amplification reaction, thereby producing an amplicon; and
c) detecting the amplicon using one or more probes selected from the group consisting of: SEQ ID NOS: 2 (coa); 5, 102 (mecA); 107, 109, 110, 111 (nuc); 8, 10, 12, 14, 16 (orfX region); 18, 21, 26, 83, 86, 89, 92 and 95 (CoNS specific marker),
wherein the detection of an amplicon is indicative of the presence of a methicillin-resistant organism or an orfX region or a coa gene or a nuc gene or a CoNS specific marker sequence in the sample.

35. A method of diagnosing a condition, syndrome or disease in a human associated with a methicillin-resistance gene or an orfX region or a coa gene or a nuc gene or a CoNS specific marker sequence, comprising contacting a denatured target from a sample with one or more probe sequences comprising a sequence selected from the group consisting of: SEQ ID NOS: 2 (coa); 5, 102 (mecA); 107, 109, 110, 111 (nuc); 8, 10, 12, 14, 16 (org region); 18, 21, 26, 83, 86, 89, 92 and 95 (CoNS specific marker) under conditions for hybridization to occur; wherein hybridization of the one or more probes to a denatured target is indicative of the presence of a methicillin-resistance gene or an org region sequence or a coa gene or a nuc gene or a CoNS specific marker sequence in the sample.
